(19) United States
(12) Patent Application Publication
(10) Pub. No.: US 2020/0123575 A1
(43) Pub. Date: Apr. 23, 2020

(54) Molecular system for cancer biology

(71) Applicant: Mohammad A. Mazed, Yorba Linda, CA (US)

(72) Inventor: Mohammad A. Mazed, Yorba Linda, CA (US)

(21) Appl. No.: 16/602,403

(22) Filed: Sep. 28, 2019

Related U.S. Application Data
(63) Continuation-in-part of application No. 16/501,943, filed on Jul. 5, 2019.
Application No. 16/501,943 is a continuation-in-part of application No. 16/350,897, filed on Jan. 29, 2019, now abandoned.
Application No. 16/350,897 is a continuation-in-part of application No. 16/350,313, filed on Oct. 30, 2018, now abandoned.
Application No. 16/350,313 is a continuation-in-part of application No. 15/998,386, filed on Aug. 14, 2018, now abandoned.
Application No. 15/998,386 is a continuation-in-part of application No. 15/932,920, filed on May 21, 2018, now abandoned.
(60) Provisional application No. 62/603,139, filed on May 19, 2017.

(51) Int. Cl. C12N 15/88; C12N 5/0784; C12N 15/11; C12N 9/22; A61K 35/15; A61K 9/50; A61K 9/51; A61K 9/127; A61K 41/00; C12N 15/113; C07K 14/705; C07K 14/74; A61K 47/69

(52) U.S. Cl. CPC C12N 15/88; C12N 2320/32; C12N 15/11; C12N 9/22; A61K 35/15; A61K 9/5052; A61K 9/501; A61K 9/51; A61K 9/1271; A61K 41/0052; C12N 15/113; C07K 14/7056; C07K 14/70539; A61K 47/6901; C12N 2510/00; C12N 2310/20; C12N 2800/80; C12N 2310/14; C12N 5/0639

(57) ABSTRACT

An engineered dendritic cell including a nanostructure, RNAi based logic circuit, a CRISPR-Cas9 system/light activated CRISPR-Cas9 system (or a modulating CRISPR-Cas9 system) and a sensing protein and/or an activating protein for enhanced interaction with a T-cell and/or a natural killer cell against a particular type of cancer cells is disclosed. The engineered dendritic cell can be ex vivo or in vivo (implanted as a biodegradable/biocompatible lymph node). Furthermore, the CRISPR-Cas9 system can be replaced by Cas9-HFI or Cas12a or a transposon-encoded CRISPR-Cas system.

### CROSS REFERENCE OF RELATED APPLICATIONS

The present application is
a continuation-in-part (CIP) of (a) U.S. Non-Provisional Patent Application with Ser. No. 16/501,943 entitled, MOLECULAR SYSTEM FOR CANCER BIOLOGY, filed on Jul. 5, 2019,
wherein (a) is a continuation-in-part (CIP) of (b) U.S. Non-Provisional Patent Application with Ser. No. 16/350,897 entitled, CRISPR-Cas9 BASED ENGINEERED/EDITED OR MODULATED STEM CELLS FOR INFLAMMATION OR PAIN, filed on Jan. 29, 2019,
wherein (b) is a continuation-in-part (CIP) of (c) U.S. Non-Provisional Patent Application with Ser. No. 16/350,313 entitled, CRISPR-Cas9 BASED MOLECULAR SYSTEM FOR INFLAMMATION OR PAIN, filed on Oct. 30, 2018,
wherein (c) is a continuation-in-part (CIP) of (d) U.S. Non-Provisional Patent Application with Ser. No. 15/998,386 entitled, CRISPR-Cas9 BASED MOLECULAR SYSTEM FOR INFLAMMATION OR PAIN, filed on Aug. 14, 2018,
wherein (d) is a continuation-in-part (CIP) of (e) U.S. Non-Provisional Patent Application with Ser. No. 15/932,920 entitled, CRISPR-Cas9 BASED MOLECULAR SYSTEM FOR INFLAMMATION OR PAIN, filed on May 21, 2018,
   wherein (e) claims priority benefit to (f) U.S. Provisional Patent Application with Ser. No. 62/603,139 entitled, “FORMULATIONS FOR INFLAMMATION/PAIN”, filed on May 19, 2017.

The entire contents of all (i) U.S. Non-Provisional Patent Applications, (ii) U.S. Provisional Patent Applications, as listed in the previous paragraph and (iii) the filed (Patent) Application Data Sheet (ADS) are hereby incorporated by reference, as if they are reproduced herein in their entirety.

### BRIEF DESCRIPTION OF THE FIGURES

FIG. 1 illustrates a progression model of Alzheimer's disease.

FIG. 2 illustrates a progression model of Parkinson's disease.

FIG. 3 illustrates a T-cell, wherein the T-cell includes (a) a sensing protein and/or an activating protein (on its surface) to detect a particular type of cancer cells and (b) a nanoshell encapsulating deoxyribonucleic acid (DNA) sequence and/or XNA sequence to encode a therapeutic protein against a particular type of cancer cells and/or one or more molecules/RNAi molecules/modified (e.g., an overexpression of certain microRNAs and/or modified ligand(s)) exosomes beneficial against a particular type of cancer cells. XNA contains genetic bases—adenine (A), thymine (T), guanine (G), cytosine (C) and uracil (U) and one or more synthetic/artificial genetic bases (e.g., α and β). The nanoshell can also include RNAi based logic circuit.

However, this configuration of a T-cell may be applicable to any diseased cells, not just to a particular type of cancer cells.

### BRIEF DESCRIPTION OF THE TABLES

Table 1 describes a pharmaceutical formulation for inflammation/pain.

Table 2 describes a pharmaceutical formulation for inflammation/pain.

### DETAILED DESCRIPTION

### Nanoshell

By way of an example and not by way of any limitation, a nanoshell (of about 5 nanometers to 500 nanometers in diameter) can be any one of the following: a boron nitride nanotube, a carbon nanotube, a Cornell-dot, a cubisome, a dendrimer (including a plant based dendrimer), a deoxyribonucleic acid origami based nanostructure, an exosome, fullerene C₆₀ (e.g., malonic acid derivative of C₆₀), a gold nanoparticle (coated for biocompatibility), a grapefruit-derived nanovector (GNV), a hollow magnetic cage molecule (e.g., Co₁₂C₆, Mn₁₂C₆ and Mn₂₄C₁₈), a metal-organic framework (MOF), a modified exosome, an iron nanoparticle, a lipidoid, a liposome, mesoporous silica, a micelle, a nanocrystal, a niosome, polysebacic acid (PSA), polysilsesquioxane (PSQ), a porous (photonic) crystal, a quantum dot, a quantum dot capped with glutathione, a ribonucleic acid (RNA) origami based nanostructure, a self-assembling peptide/protein, a silk-fibroin nanoparticle, a solid-lipid nanoparticle, a spherical nucleic acid (SNA), synthasome, a tubular/tetrahedral structure fabricated/constructed utilizing deoxyribonucleic acid/ribonucleic acid/XNA origami based nanostructure, a virus (e.g., tobacco mosaic virus), zein (plant) protein, a XNA origami based nanostructure, a worm-shaped nanoparticle and a zeolite-L-nanocrystal.

The deoxyribonucleic acid origami based nanostructure/ribonucleic acid origami based nanostructure/XNA Origami based nanostructure can be three-dimensional in shape.

Silk fibroin is biodegradable and biocompatible.

Additionally, a nanoshell can made of synthetic lipids including disulfide bonds in the fatty chain. Furthermore, when the nanoshell (of synthetic lipids including disulfide bonds in the fatty chain) enters a cell, the environment within the cell breaks open the disulfide bond to disassemble the nanoshell and the contents of the nanoshell can be quickly and efficiently released into the cell.

Furthermore, a nanoshell can be a nanobot or a nanomotor in some biological applications.

### Macroshell

By way of an example and not by way of any limitation, a macroshell (of about 1 micron to 50 microns in diameter) can be an approximately spherically shaped structure (e.g., with composition of polycaprolactone polymer/hydrogel).

### Bioavailability, Sustained (Controlled) Delivery & Disintegration

To increase bioavailability of any bioactive compound (of the Table 1 and Table 2), microemulsion/nanoemulsion or microsuspension/nanosuspension of the bioactive compound can be utilized.

To prevent any dissolution/disintegration in the gastric (acidic) environment of any bioactive compound (of the Table 1 and Table 2), the macroshell or the nanoshell itself can be enteric coated by a polymer barrier.

To increase bioavailability and/or sustained (controlled) delivery of the entire pharmaceutical formulation (of the Table 1 and Table 2), microencapsulation of the entire pharmaceutical formulation (of the Table 1 and Table 2) or nanoencapsulation of any bioactive compound (of the Table 1 and Table 2) in a nanoshell can be utilized.

The macroshell or the nanoshell can include an immune shielding surface (as it is discussed in later paragraphs).

### Immune Shielding Surface of a Nanoshell

A polymer membrane (e.g., polyethylene glycol polymer) can be utilized, as an immune shielding surface on a nanoshell.

Alternatively, a red blood (natural/synthetic/three-dimensionally bio-inkjet printed red blood) cell membrane can be utilized, as an immune shielding surface on the nanoshell.

Alternatively, a combination of a polymer membrane and a red blood cell membrane can be utilized, as an immune shielding surface on the nanoshell.

Alternatively, the nanoshell can bind/couple (e.g., chemically bind/couple) or attach with a red blood cell (about 8 microns in diameter) to act as an immune shielding surface on the nanoshell. A red blood cell is about 8 microns in diameter.

Alternatively, the nanoshell can bind/couple (e.g., chemically bind/couple) or attach with a white blood cell (about 5 microns to 17 microns in diameter) to act as an immune shielding surface on the nanoshell.

### Immune Shielding Surface of a Macroshell

A polymer membrane (e.g., polyethylene glycol polymer) can be utilized, as an immune shielding surface on the macroshell.

Alternatively, a red blood (natural/synthetic/three-dimensionally bio-inkjet printed red blood) cell membrane can be utilized, as an immune shielding surface on the macroshell.

Alternatively, a combination of a polymer membrane and a red blood cell membrane can be utilized, as an immune shielding surface on the macroshell.

Alternatively, the nanoshell can bind/couple (e.g., chemically bind/couple) or attach with a red blood cell to act as an immune shielding surface on the macroshell.

Alternatively, the nanoshell can bind/couple (e.g., chemically bind/couple) or attach with a white blood cell to act as an immune shielding surface on the macroshell.

Details of an immune shielding surface have been disclosed in U.S. Non-Provisional patent application Ser. No. 13/135,832 entitled “CHEMICAL COMPOSITION AND ITS DELIVERY FOR LOWERING THE RISKS OF ALZHEIMER'S, CARDIOVASCULAR AND TYPE-2 DIABETES DISEASES”, filed on Jul. 15, 2011.

TABLE 1
Pharmaceutical Formulation

| | WT % +/− 20% |
|---|---|
| Botanical | |
| Boswellia serrata Extract | 20% |
| Chemical | |
| Astaxanthin | 2.5% |
| Capsaicin | 5% |
| CBD (Or CBD Pro-Drug) | 40% |
| Chondroitin Sulfate | 5% |
| Glucosamine Hydrochloride Or Glucosamine Sulfate | 5% |
| Hyaluronic Acid | 5% |
| Lidocaine Hydrochloride | 2.5% |
| Methylsufonlymethane (MSM) | 5% |
| Menthol | 5% |
| S-Adenosyl Methionine (SAM) | 5% |
| Total | 100% |

TABLE 2
Pharmaceutical Formulation

| | WT % +/− 20% |
|---|---|
| Botanical | |
| Boswellia serrata Extract | 20% |
| Chemical | |
| Capsaicin | 5% |
| CBD (Or CBD Pro-Drug) | 42.5% |
| Chondroitin Sulfate | 5% |
| Glucosamine Hydrochloride Or Glucosamine Sulfate | 5% |
| Hyaluronic Acid | 5% |
| Lidocaine Hydrochloride | 2.5% |
| Methylsufonlymethane (MSM) | 5% |
| Menthol | 5% |
| S-Adenosyl Methionine (SAM) | 5% |
| Total | 100% |

Cannabidiol (CBD)'s molecular structure is described below.

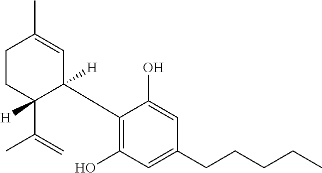 [SMILES: C=C(C)[C@@H]1CCC(C)=C[C@H]1c1c(O)cc(CCCCC)cc1O]

CBD or CBD pro-drug can be replaced by Perrottetinene (PET) and its molecular structure is described below.

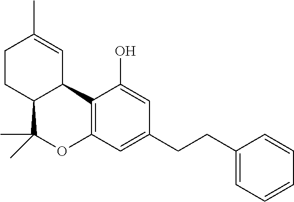 [SMILES: CC1=C[C@H]2c3c(O)cc(CCc4ccccc4)cc3OC(C)(C)[C@H]2CC1]

Any bioactive compound of the Table 1 and Table 2 loaded with silk fibroin-all encapsulated/caged in a macroshell (with an immune shielding surface) or alternatively, the entire pharmaceutical formulation of the Table 1 and Table 2 loaded with silk fibroin-all encapsulated/caged in a macroshell (with an immune shielding surface) can provide an effective long and lasting relief of inflammation or pain (e.g., via a tablet/patch/injection).

Any bioactive compound of the Table 1 and Table 2 loaded with (a) silk fibroin and/or (b) platelet-rich plasma (PRP)—all encapsulated/caged in a macroshell (with an immune shielding surface) or alternatively, the entire pharmaceutical formulation of the Table 1 and Table 2 loaded with (a) silk fibroin and/or (b) platelet-rich plasma (PRP)—all encapsulated/caged in a macroshell (with an immune shielding surface) can provide an effective long and lasting relief of inflammation or pain (e.g., via a tablet/patch/injection).

Any bioactive compound of the Table 1 and Table 2 loaded with (a) silk fibroin and/or (b) platelet-rich plasma (PRP) and/or (c) tissue graft from a stem cell (e.g., a pluripotent stem cell/three-dimensional (3-D) bio-inkjet printed stem cell)—all encapsulated/caged in a macroshell (with an immune shielding surface) or alternatively, the entire pharmaceutical formulation of the Table 1 and Table 2 loaded with (a) silk fibroin and/or (b) platelet-rich plasma (PRP) and/or (c) tissue graft from a stem cell-all encapsulated/caged in a macroshell (with an immune shielding surface) can provide an effective long and lasting relief of inflammation or pain or tissue regeneration (e.g., via a tablet/patch/injection).

Any bioactive compound of the Table 1 and Table 2 loaded with (a) silk fibroin and/or (b) platelet-rich plasma (PRP) and/or (c) tissue graft from an engineered/edited stem cell or a modulated stem cell-all encapsulated/caged in a macroshell (with an immune shielding surface) or alternatively, the entire pharmaceutical formulation of the Table 1 and Table 2 loaded with (a) silk fibroin and/or (b) platelet-rich plasma (PRP) and/or (c) tissue graft from an engineered/edited stem cell or a modulated stem cell-all encapsulated/caged in a macroshell (with an immune shielding surface) can provide an effective long and lasting relief of inflammation or pain or tissue regeneration (e.g., via a tablet/patch/injection).

### CRISPR-Cas9 System/Light Activated CRISPR-Cas9 System

Cas9 is an RNA-directed deoxyribonucleic acid-binding protein, guided by a single guide RNA. By inactivating its nuclease activity, coupling the protein to other effector domains and choosing an appropriate guide sequence, it can direct activities in a specific part of a genome.

CRY2 and CIB1 are plant proteins. In response to light, CRY2 undergoes a conformational change that allows it to interact with CIB1. In a light activated CRISPR-Cas9 system, CRY2 is fused to the transactivation domain (either p65 or VP64) and C1B1 is fused to dCas9—the deactivated Cas9 nuclease from CRISPR. The light activated CRISPR-Cas9 system can enable precise spatial and/or temporal control by light (e.g., blue light) and can direct activities in a specific part of a genome.

Generally, a CRISPR-Cas9 system or a light activated CRISPR-Cas9 system can remove a first defective gene and add/insert/replace a second gene, which may be beneficial against a particular type of diseased cells.

### Modulating CRISPR-Cas9 System

A modulating CRISPR-Cas9 system includes Cas9, a short single guide RNA, a transcriptional activator and an adeno-associated virus (AAV).

The modulating CRISPR-Cas9 does not remove or add/insert/replace any gene. Rather it can activate an expression of one or more genes beneficial against a particular type of diseased cells.

Various gene editing methods has been disclosed in “OPTICAL BIOMODULE FOR DETECTION OF DISEASES”, filed on Oct. 29, 2012, wherein U.S. Non-Provisional patent application Ser. No. 13/663,376 resulted in an issuance of a U.S. Pat. No. 9,557,271 on Jan. 31, 2017.

### Engineered/Edited Stem Cell

The nanoshell (encapsulating/caging the CRISPR-Cas9 system or the light activated CRISPR-Cas9 system) can deliver the CRISPR-Cas9 system or the light activated CRISPR-Cas9 system into a stem cell by an external stimulus (e.g., heat/light/pH/receptor binding).

Alternatively, the above nanoshell can be decorated with one or more targeting ligands to recognize/match/bind with specific biological receptors on a stem cell to deliver the CRISPR-Cas9 system or the light activated CRISPR-Cas9 system into a stem cell.

The nanoshell (encapsulating/caging the CRISPR-Cas9 system or the light activated CRISPR-Cas9 system) can include one or more molecules/RNAi molecules/modified (e.g., an overexpression of certain microRNAs and/or modified ligand(s)) exosomes beneficial against a particular type of diseased cells

The nanoshell (encapsulating/caging the CRISPR-Cas9 system or the light activated CRISPR-Cas9 system) can include or bind/couple (e.g., chemically bind/couple) with a sensing protein and/or an activating protein to detect a molecular event that occurs within a particular type of diseased cells.

### Modulated Stem Cell

The modulating CRISPR-Cas9 system (inserted) into a stem cell activates an expression of one or more genes beneficial against a particular diseased cells-thus enabling a modulated stem cell.

The modulated stem cell can include or bind/couple (e.g., chemically bind/couple) with a sensing protein and/or an activating protein to detect a molecular event within a particular type of diseased cells.

The modulated stem cell can include with one or more molecules/RNAi molecules/modified (e.g., an overexpression of certain microRNAs and/or modified ligand(s)) exosomes beneficial against a particular type of diseased cells

### Encapsulation of Engineered/Edited Stem Cell or Modulated Stem Cell

The engineered/edited or the modulated stem cell can be encapsulated/caged in a macroshell.

The macroshell can include an immune shielding surface (details of an immune shielding surface were discussed in previous paragraphs).

Application to Treatment of Inflammation without an Engineered/Edited or Modulated Stem Cell

### CRISPR-Cas9 System/Light Activated CRISPR-Cas9 System

The CRISPR-Cas9 system or the light activated CRISPR-Cas9 system can be encapsulated/caged in the nanoshell, wherein the nanoshell includes an immune shielding surface, wherein the CRISPR-Cas9 system or the light activated CRISPR-Cas9 system can remove a first gene (e.g., a defective/mutated gene) responsible for inflammation and add/insert/replace a second gene (including genetic bases A (adenine), C (cytosine), T (thymine), G (guanine) and/or one or more synthetic/artificial genetic bases (e.g., α and β)) to express/release a molecule (e.g., an antibody/hormone/protein) to reduce inflammation (e.g., tumor necrosis factor-alpha (TNF-alpha) inhibitor) or pain.

Inserting genes in the point cleaved by CRISPR-Cas9 system may require other proteins. A cell may contain or can be configured to contain such proteins. Thus, a complementary sequence on the ends of the insertion sequence may be needed to add/insert/replace a second gene (including genetic bases A (adenine), C (cytosine), T (thymine), G (guanine) and/or one or more synthetic/artificial genetic bases (e.g., α and β)) inside a cell.

The nanoshell (encapsulating/caging the CRISPR-Cas9 system or the light activated CRISPR-Cas9 system) can include or bind/couple (e.g., chemically bind/couple) with a sensing protein and/or an activating protein to detect a molecular event of inflammation within a particular type of inflamed cells.

The nanoshell (encapsulating/caging the CRISPR-Cas9 system or the light activated CRISPR-Cas9 system) can include or bind/couple (e.g., chemically bind/couple) with one or more molecules/RNAi molecules/modified (e.g., an overexpression of certain microRNAs and/or modified ligand(s)) exosomes beneficial against inflammation.

### Modulating CRISPR-Cas9 System

The modulating CRISPR-Cas9 system can activate an expression of one or more genes beneficial against inflammation.

The modulating CRISPR-Cas9 system can be encapsulated/caged in the nanoshell, wherein the nanoshell includes an immune shielding surface.

The nanoshell (encapsulating/caging the modulating CRISPR-Cas9 system) can include or bind/couple (e.g., chemically bind/couple) with a sensing protein and/or an activating protein to detect a molecular event of inflammation within a particular type of inflamed cells.

The nanoshell (encapsulating/caging the modulating CRISPR-Cas9 system) can include or bind/couple (e.g., chemically bind/couple) can include or bind/couple (e.g., chemically bind/couple) with one or more molecules/RNAi molecules/modified (e.g., an overexpression of certain microRNAs and/or modified ligand(s)) exosomes beneficial against inflammation.

Application to Treatment of Inflammation without an Engineered/Edited or Modulated Stem Cell

Deoxyribonucleic acids can be coupled with a metal (e.g., gold) nanorod. Upon infrared light activation, deoxyribonucleic acids can be decoupled from the metal nanorod due to heating. Upon Deoxyribonucleic acids-receptors coupling, stem cells can migrate to a site of inflammation or pain for tissue regeneration.

Application to Treatment of Inflammation with an Engineered/Edited or Modulated Stem Cell

### Engineered/Edited Stem Cell

The CRISPR-Cas9 system or the light activated CRISPR-Cas9 system via the nanoshell can be inserted into a stem cell to remove a first gene responsible for inflammation or pain and then add/insert/replace a second gene (including genetic bases A (adenine), C (cytosine), T (thymine), G (guanine) and/or one or more synthetic/artificial genetic bases (e.g., α and β)) that expresses/releases a molecule to reduce inflammation or pain (and/or enhance regeneration of tissues)—thus enabling an engineered/edited stem cell.

Inserting genes in the point cleaved by CRISPR-Cas9 system may require other proteins. An engineered or edited stem cell may contain or can be configured to contain such proteins. Thus, a complementary sequence on the ends of the insertion sequence may be needed to add/insert/replace a second gene (including genetic bases A (adenine), C (cytosine), T (thymine), G (guanine) and/or one or more synthetic/artificial genetic bases (e.g., α and β)) inside a stem cell.

The nanoshell (encapsulating/caging the CRISPR-Cas9 system or the light activated CRISPR-Cas9 system) can include or bind/couple (e.g., chemically bind/couple) with a sensing protein and/or an activating protein to detect a molecular event of inflammation within a particular type of inflamed cells. Furthermore, the nanoshell includes an immune shielding surface.

The nanoshell (encapsulating/caging the CRISPR-Cas9 system or the light activated CRISPR-Cas9 system) can include or bind/couple (e.g., chemically bind/couple) with one or more molecules/RNAi molecules/modified (e.g., an overexpression of certain microRNAs and/or modified ligand(s)) exosomes beneficial against inflammation. Furthermore, the nanoshell can include an immune shielding surface.

### Modulated Stem Cell

The modulating CRISPR-Cas9 system inserted into a stem cell. The modulating CRISPR-Cas9 system can activate an expression of one or more genes beneficial against inflammation-thus enabling a modulated stem cell.

The modulated stem cell can include or bind/couple (e.g., chemically bind/couple) with a sensing protein and/or an activating protein to detect a molecular event of inflammation within a particular type of inflamed cells

The modulated stem cell can include with one or more molecules/RNAi molecules/modified (e.g., an overexpression of certain microRNAs and/or modified ligand(s)) exosomes beneficial against inflammation.

Application to Treatment of Alzheimer's Disease without an Engineered/Edited or Modulated Stem Cell

### CRISPR-Cas9 System/Light Activated CRISPR-Cas9 System

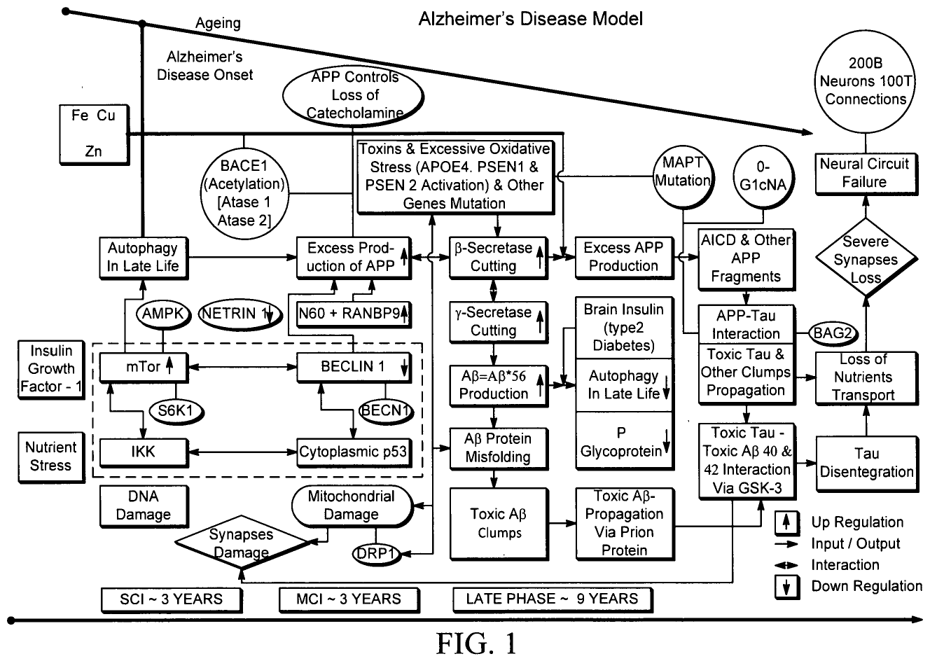

The CRISPR-Cas9 system or the light activated CRISPR-Cas9 system can be encapsulated/caged in the nanoshell, wherein the nanoshell includes an immune shielding surface, wherein the CRISPR-Cas9 system or the light activated CRISPR-Cas9 system can remove a first gene (e.g., defective/mutated ApoE4 gene) responsible for Alzheimer's disease and add/insert/replace a second gene (including genetic bases A (adenine), C (cytosine), T (thymine), G (guanine) and/or one or more synthetic/artificial genetic bases (e.g., α and β)) to express/release a molecule (e.g., an antibody/hormone/protein) to reduce accumulation of amyloid beta protein and/or tau protein. (FIG. 1).

Inserting genes in the point cleaved by CRISPR-Cas9 system may require other proteins. A cell may contain or can be configured to contain such proteins. Thus, a complementary sequence on the ends of the insertion sequence may be needed to add/insert/replace a second gene (including genetic bases A (adenine), C (cytosine), T (thymine), G (guanine) and/or one or more synthetic/artificial genetic bases (e.g., α and β)) inside a cell.

The nanoshell (encapsulating/caging the CRISPR-Cas9 system or the light activated CRISPR-Cas9 system) can include or bind/couple (e.g., chemically bind/couple) with a sensing protein and/or an activating protein to detect a molecular event within a particular type of neurons.

The nanoshell (encapsulating/caging the CRISPR-Cas9 system or the light activated CRISPR-Cas9 system) can include or bind/couple (e.g., chemically bind/couple) with one or more molecules/RNAi molecules/modified (e.g., an overexpression of certain microRNAs and/or modified ligand(s)) exosomes beneficial against Alzheimer's disease.

The nanoshell can include or bind/couple (e.g., chemically couple) with an antibody or surface modification with a functional ligand (e.g., lactoferrin/transferrin) and/or a carrier molecule (e.g., glutathione/glucose) to cross the blood-brain barrier (BBB).

### Modulating CRISPR-Cas9 System

The modulating CRISPR-Cas9 system can activate an expression of one or more genes beneficial against Alzheimer's disease.

The modulating CRISPR-Cas9 system can be encapsulated/caged in the nanoshell, wherein the nanoshell includes an immune shielding surface.

The nanoshell (encapsulating/caging the modulating CRISPR-Cas9 system) can include or bind/couple (e.g., chemically bind/couple) with a sensing protein and/or an activating protein to detect a molecular event of Alzheimer's disease within a particular type of neurons.

The nanoshell (encapsulating/caging the modulating CRISPR-Cas9 system) can include or bind/couple (e.g., chemically bind/couple) can include or bind/couple (e.g., chemically bind/couple) with one or more molecules/RNAi molecules/modified (e.g., an overexpression of certain microRNAs and/or modified ligand(s)) exosomes beneficial against Alzheimer's disease.

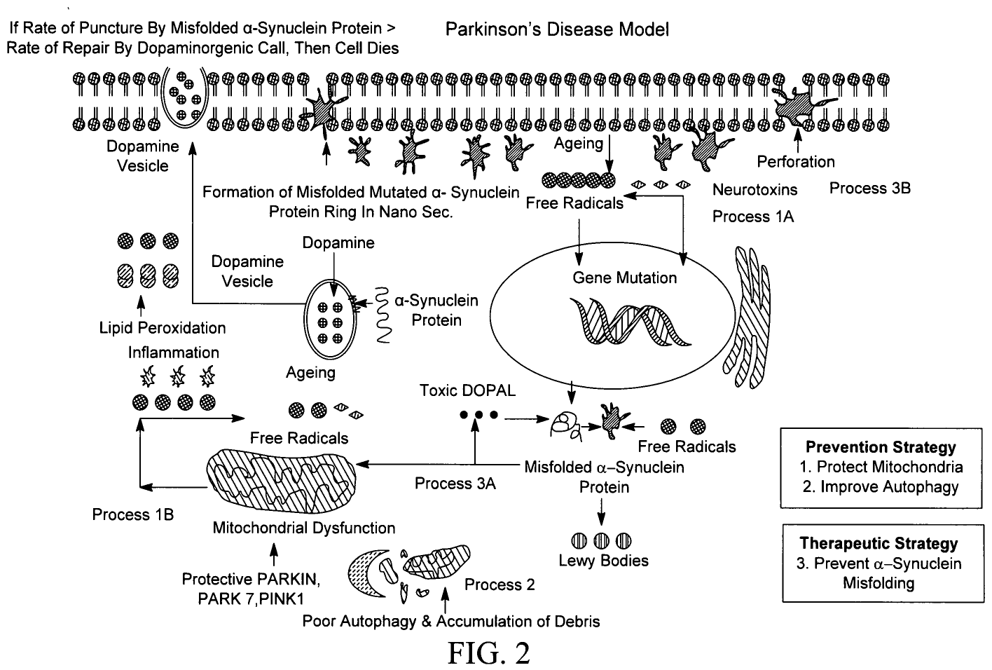

This approach can be applicable to other neurodegenerative diseases (e.g., Parkinson's disease (FIG. 2)/dementia with Lewy bodies).

Application to Treatment of Alzheimer's Disease with Engineered/Edited or Modulated Stem Cell

### Engineered/Edited Stem Cell

A stems cell such as mesenchymal stem cell can cross the blood-brain barrier. The CRISPR-Cas9 system or the light activated CRISPR-Cas9 system via the nanoshell can be inserted into a mesenchymal stem cell to remove a first gene responsible for Alzheimer's disease and then add/insert/replace a second gene (including genetic bases A (adenine), C (cytosine), T (thymine), G (guanine) and/or one or more synthetic/artificial genetic bases (e.g., α and β)) that expresses/releases a molecule (e.g., an antibody/hormone/protein) for reduction in accumulation of amyloid beta protein and/or tau protein. (FIG. 1)—thus enabling an engineered/edited mesenchymal stem cell.

Inserting genes in the point cleaved by CRISPR-Cas9 system may require other proteins. An engineered or edited mesenchymal stem cell may contain or can be configured to contain such proteins. Thus, a complementary sequence on the ends of the insertion sequence may be needed to add/insert/replace a second gene (including genetic bases A (adenine), C (cytosine), T (thymine), G (guanine) and/or one or more synthetic/artificial genetic bases (e.g., α and β)) inside a stem cell.

The nanoshell (encapsulating/caging the CRISPR-Cas9 system or the light activated CRISPR-Cas9 system) can include or bind/couple (e.g., chemically bind/couple) with a sensing protein and/or an activating protein to detect a molecular event of Alzheimer's disease within a particular type of neurons. Furthermore, the nanoshell includes an immune shielding surface.

The nanoshell (encapsulating/caging the CRISPR-Cas9 system or the light activated CRISPR-Cas9 system) can include or bind/couple (e.g., chemically bind/couple) with one or more molecules/RNAi molecules/modified (e.g., an overexpression of certain microRNAs and/or modified ligand(s)) exosomes beneficial against Alzheimer's disease. Furthermore, the nanoshell can include an immune shielding surface.

### Modulated Stem Cell

The modulating CRISPR-Cas9 system inserted into a mesenchymal stem can activate an expression of one or more genes beneficial against Alzheimer's disease-thus enabling a modulated mesenchymal stem cell.

The modulated stem cell can include or bind/couple (e.g., chemically bind/couple) with a sensing protein and/or an activating protein to detect a molecular event of Alzheimer's disease within a particular type of neurons.

The modulated stem cell can include one or more molecules/RNAi molecules/modified (e.g., an overexpression of certain microRNAs and/or modified ligand(s)) exosomes beneficial against Alzheimer's disease. The modulated stem cell can transfer healthy mitochondria to damaged neurons for neuroregeneration.

Application to Treatment of Brain Cancer without an Engineered/Edited or Modulated Stem Cell

### CRISPR-Cas9 System/Light Activated CRISPR-Cas9 System

The CRISPR-Cas9 system or the light activated CRISPR-Cas9 system can be encapsulated/caged in the nanoshell, wherein the nanoshell includes an immune shielding surface, wherein the CRISPR-Cas9 system or the light activated CRISPR-Cas9 system can remove a first gene (e.g., a defective/mutated gene) responsible for brain cancer and add/insert/replace a second gene (including genetic bases A (adenine), C (cytosine), T (thymine), G (guanine) and/or one or more synthetic/artificial genetic bases (e.g., a and β)) to express/release a molecule (e.g., an antibody/hormone/protein) for apoptosis of a brain cancer cell and/or reduction of brain cancer growth (e.g., blocking an enzyme to inhibit self-regenerating capabilities glioma stem cell (GSC)).

Inserting genes in the point cleaved by CRISPR-Cas9 system may require other proteins. A cell may contain or can be configured to contain such proteins. Thus, a complementary sequence on the ends of the insertion sequence may be needed to add/insert/replace a second gene (including genetic bases A (adenine), C (cytosine), T (thymine), G (guanine) and/or one or more synthetic/artificial genetic bases (e.g., α and β)) inside a cell.

The nanoshell (encapsulating/caging the CRISPR-Cas9 system or the light activated CRISPR-Cas9 system) can include or bind/couple (e.g., chemically bind/couple) a sensing protein and/or an activating protein to detect a molecular event within a particular type of brain cancer cells.

The nanoshell (encapsulating/caging the CRISPR-Cas9 system or the light activated CRISPR-Cas9 system) can include or bind/couple (e.g., chemically bind/couple) with one or more molecules/RNAi molecules/modified (e.g., an overexpression of certain microRNAs and/or modified ligand(s)) exosomes beneficial against a particular type of brain cancer cells.

The nanoshell may include or bind/couple (e.g., chemically couple) with an immune-checkpoint inhibitor molecule.

The nanoshell (encapsulating/caging the CRISPR-Cas9 system or the light activated CRISPR-Cas9 system) can include or bind/couple (e.g., chemically bind/couple) with biocompatible magnetic nanoparticles (e.g., Zn—Co—Cr-Ferrite nanoparticles or mn-co-Ferrite nanoparticles). Such biocompatible magnetic nanoparticles can be heated by a low magnetic field.

The nanoshell can include or bind/couple (e.g., chemically couple) with an antibody or surface modification with a functional ligand (e.g., lactoferrin/transferrin) and/or a carrier molecule (e.g., glutathione/glucose) to cross the blood-brain barrier (BBB).

Combining the above approach with photodynamic therapy (PDT) delivered to a particular type of brain cancer cells through a stereotactically placed optical fiber(s) can enhance therapeutic outcome.

### Modulating CRISPR-Cas9 System

The modulating CRISPR-Cas9 system can activate an expression of one or more genes beneficial against brain cancer.

The modulating CRISPR-Cas9 system can be encapsulated/caged in the nanoshell, wherein the nanoshell includes an immune shielding surface.

The nanoshell (encapsulating/caging the modulating CRISPR-Cas9 system) can include or bind/couple (e.g., chemically bind/couple) with a sensing protein and/or an activating protein to detect a molecular event within a particular type of brain cancer cells.

The nanoshell (encapsulating/caging the modulating CRISPR-Cas9 system) can include or bind/couple (e.g., chemically bind/couple) can include or bind/couple (e.g., chemically bind/couple) with one or more molecules/RNAi molecules/modified (e.g., an overexpression of certain microRNAs and/or modified ligand(s)) exosomes beneficial against brain cancer.

Combining the above approach with photodynamic therapy (PDT) delivered to a particular type of brain cancer cells through a stereotactically placed optical fiber(s) can enhance therapeutic outcome.

Application to Treatment of Brain Cancer With an Engineered/Edited Or Modulated Stem Cell

### Engineered/Edited Stem Cell

A stems cell such as mesenchymal stem cell can cross the blood-brain barrier. The CRISPR-Cas9 system or the light activated CRISPR-Cas9 system via the nanoshell can be inserted into a mesenchymal stem cell to remove a first gene responsible for a first gene (e.g., a defective/mutated gene) responsible for brain cancer and then add/insert/replace a second gene (including genetic bases A (adenine), C (cytosine), T (thymine), G (guanine) and/or one or more synthetic/artificial genetic bases (e.g., α and β)) that expresses/releases a molecule (e.g., an antibody/hormone/protein) for apoptosis of a brain cancer cell and/or reduction of brain cancer growth (e.g., blocking an enzyme to inhibit self-regenerating capabilities glioma stem cell (GSC)).

Inserting genes in the point cleaved by CRISPR-Cas9 system may require other proteins. An engineered or edited mesenchymal stem cell may contain or can be configured to contain such proteins. Thus, a complementary sequence on the ends of the insertion sequence may be needed to add/insert/replace a second gene (including genetic bases A (adenine), C (cytosine), T (thymine), G (guanine) and/or one or more synthetic/artificial genetic bases (e.g., α and β)) inside a stem cell.

The nanoshell (encapsulating/caging the CRISPR-Cas9 system or the light activated CRISPR-Cas9 system) can include or bind/couple (e.g., chemically bind/couple) with a sensing protein and/or an activating protein to detect a molecular event within a particular type of brain cancer cells.

The nanoshell (encapsulating/caging the CRISPR-Cas9 system or the light activated CRISPR-Cas9 system) can include or bind/couple (e.g., chemically bind/couple) with one or more molecules/RNAi molecules/modified (e.g., an overexpression of certain microRNAs and/or modified ligand(s)) exosomes beneficial against a particular type of brain cancer cells.

The nanoshell may include or bind/couple (e.g., chemically couple) with an immune-checkpoint inhibitor molecule.

The nanoshell (encapsulating/caging the CRISPR-Cas9 system or the light activated CRISPR-Cas9 system) can include or bind/couple (e.g., chemically bind/couple) with biocompatible magnetic nanoparticles (e.g., Zn—Co—Cr-Ferrite nanoparticles or mn-co-Ferrite nanoparticles). Such biocompatible magnetic nanoparticles can be heated by a low magnetic field.

Combining the above approach with photodynamic therapy (PDT) delivered to a particular type of brain cancer cells through a stereotactically placed optical fiber(s) can enhance therapeutic outcome.

### Modulated Stem Cell

The modulating CRISPR-Cas9 system inserted into a mesenchymal stem can activate an expression of one or more genes beneficial against brain cancer-thus enabling a modulated mesenchymal stem cell.

The modulated stem cell can include or bind/couple (e.g., chemically bind/couple) with a sensing protein and/or an activating protein to detect a molecular event within a particular type of brain cancer cells.

The modulated stem cell can include one or more molecules/RNAi molecules/modified (e.g., an overexpression of certain microRNAs and/or modified ligand(s)) exosomes beneficial against a particular type of brain cancer cells.

The nanoshell may include or bind/couple (e.g., chemically couple) with an immune-checkpoint inhibitor molecule.

The nanoshell (encapsulating/caging the CRISPR-Cas9 system or the light activated CRISPR-Cas9 system) can include or bind/couple (e.g., chemically bind/couple) with biocompatible magnetic nanoparticles (e.g., Zn—Co—Cr-Ferrite nanoparticles or mn-co-Ferrite nanoparticles). Such biocompatible magnetic nanoparticles can be heated by a low magnetic field.

Combining the above approach with photodynamic therapy (PDT) delivered to a particular type of brain cancer cells through a stereotactically placed optical fiber(s) can enhance therapeutic outcome.

Application to Treatment of Cancer without an Engineered/Edited or Modulated Stem Cell CRISPR-Cas9 System/Light Activated CRISPR-Cas9 System

Calcium is generally increased in a cancer cell. Calcium stimulates the secretion of exosomes due to calcium-binding protein called Munc13-4. Furthermore, Munc13-4 protein levels are often elevated in breast, pancreatic and lung cancers. Munc13-4 protein couples (e.g., chemically couples) with Rab11 protein to develop multivesicular bodies for fusing with the plasma membrane and releasing exosomes.

Exosomes released from cancer cells carry MT1-MMP enzyme, which can degrade in the extracellular matrix around cancer cells, by dispersing a secondary metastatic cancer cells.

The CRISPR-Cas9 system or the light activated CRISPR-Cas9 system can be encapsulated/caged in the nanoshell, wherein the nanoshell includes an immune shielding surface, wherein the CRISPR-Cas9 system or the light activated CRISPR-Cas9 system can remove a first gene (e.g., a defective/mutated gene responsible for Munc13-4 protein) responsible for cancer and add/insert/replace a second gene (including genetic bases A (adenine), C (cytosine), T (thymine), G (guanine) and/or one or more synthetic/artificial genetic bases (e.g., α and β)) to express/release a molecule (e.g., an antibody/hormone/protein) for reduce cancer growth (e.g., a gene for turning on gulonolactone oxidase production or activation of p53 gene responsible for apoptosis of a particular type of cancer cells.

Inserting genes in the point cleaved by CRISPR-Cas9 system may require other proteins. A cell may contain or can be configured to contain such proteins. Thus, a complementary sequence on the ends of the insertion sequence may be needed to add/insert/replace a second gene (including genetic bases A (adenine), C (cytosine), T (thymine), G (guanine) and/or one or more synthetic/artificial genetic bases (e.g., α and β)) inside a cell.

The nanoshell (encapsulating/caging the CRISPR-Cas9 system or the light activated CRISPR-Cas9 system) can include or bind/couple (e.g., chemically bind/couple) with a sensing protein and/or an activating protein to detect a molecular event within a particular type of cancer cells.

The nanoshell (encapsulating/caging the CRISPR-Cas9 system or the light activated CRISPR-Cas9 system) can include or bind/couple (e.g., chemically bind/couple) with one or more molecules/RNAi molecules/modified (e.g., an overexpression of certain microRNAs and/or modified ligand(s)) exosome beneficial against a particular type of cancer cells.

The nanoshell may include or bind/couple (e.g., chemically couple) with an immune-checkpoint inhibitor molecule.

The nanoshell (encapsulating/caging the CRISPR-Cas9 system or the light activated CRISPR-Cas9 system) can include or bind/couple (e.g., chemically bind/couple) with biocompatible magnetic nanoparticles (e.g., Zn—Co—Cr-Ferrite nanoparticles or mn-co-Ferrite nanoparticles). Such biocompatible magnetic nanoparticles can be heated by a low magnetic field.

The nanoshell (encapsulating/caging the CRISPR-Cas9 system or the light activated CRISPR-Cas9 system) can include or bind/couple (e.g., chemically bind/couple) with biocompatible magnetic nanoparticles (e.g., Zn—Co—Cr-Ferrite nanoparticles or mn-co-Ferrite nanoparticles). Such biocompatible magnetic nanoparticles can be heated by a low magnetic field.

The nanoshell can include or couple (e.g., chemically couple) with a separate nanosystem.

The separate nanosystem can be coated with lipid oleate and DOTAP to enhance its interaction and penetration into a particular type of cancer cells.

The separate nanosystem can include a tube shaped nanoscaled deoxyribonucleic acid cargo/nanoscaled metal (e.g., gold) rod (which is about 35 nanometers long and about 10 nanometers in radius).

The tube shaped nanoscaled deoxyribonucleic acid cargo/nanoscaled metal rod can be coupled/chemically coupled with a heat shock protein and/or TRAIL protein.

Furthermore, the tube shaped nanoscaled deoxyribonucleic acid cargo/nanoscaled metal rod can include or couple (e.g., chemically couple) with a biocompatible metal (e.g., gold) nanoparticle, a nanoshell X and a nanoshell Y by strands of a biological material (e.g., apatmers/deoxyribonucleic acids/ribonucleic acids/XNAs).

The biocompatible metal nanoparticle, the nanoshell X and the nanoshell Y includes an immune shielding surface.

About fifteen (15) X/Y nanoshells can be coupled.

The strands of the biological material can include or couple (e.g., chemically couple) with one or more molecules beneficial against a particular type of cancer cells (e.g., a synthetic p53 protein(s)/synthetic version of a cannabis flavonoid(s)) and/or an RNAi molecule(s) and/or a photosensitizer molecule(s).

For example, a cannabis flavonoid is any of these molecules: Cannflavin A, Cannflavin B, Cannflavin C, Chrysoeriol, Cosmosiin and Flavocannabiside—but not limited to these examples.

The chemical structure of Cannflavin A is illustrated below:

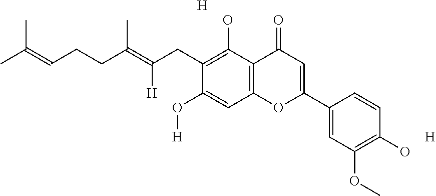

The chemical structure of Cannflavin B is illustrated below:

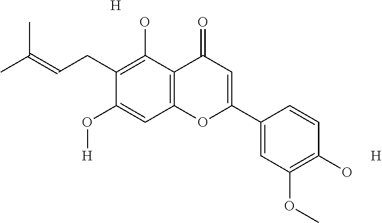

It should be noted that Cannflavin B causes apoptosis and enhances the effectiveness of common chemo- and radiation therapies.

The nanoshell X can include or couple (e.g., chemically couple) with a near-infrared fluorescent polymer to visualize its accumulation at the target location of a particular type of cancer cells.

The nanoshell Y can be an upconverting nanoshell which converts a (continuous wave/pulsed) laser light of near-infrared wavelength into a (continuous wave/pulsed) laser light of visible wavelength.

The separate nanosystem can include one or more liposomes. Each liposome can encapsulate bioactive molecules/drugs and magnetic nanoparticles. Upon heating by a high frequency and low intensity magnetic field, the liposome can undergo a phase change from solid to liquid—thus releasing the bioactive molecules/drugs at a time t=0. But, when the high frequency and low intensity magnetic field is turned off, the lipids re-solidify due to reverse phase change, preventing any release of the bioactive molecules/drugs at a time t=t.

The separate nanosystem can further include or couple (e.g., chemically couple) with cerium fluoride nanoparticles and/or hafnium oxide.

Cerium fluoride nanoparticles can release reactive oxygen species upon activated/stimulated by X-rays of a (suitable) dose to destroy a particular type of cancer cells.

Hafnium oxide can generate electrons when exposed to X-rays of a (suitable) dose to destroy a particular type of cancer cells.

Chimeric antigen receptor (CAR) is produced by splicing together the gene for an antibody that recognizes a tumor antigen and the gene for a receptor that sits on the surface of the T-cells.

CAR natural killer (NK) cells may be safer without many side effects, faster to produce and cheaper. CAR natural killer cells can be less vulnerable to cancer cells' tricks for avoiding attacks, as natural killer cells generally rely on other receptors to recognize a particular type of cancer cells and they may detect a particular type of cancer cells, even when a particular type of cancer cells modifies the antigens.

There may be multiple doses of CAR natural killer cells, as opposed to a single dose of the T-cells.

### Engineered T-cell/Natural Killer Cell/CAR T-cell/CAR Natural Killer Cell

The CRISPR-Cas9 system or the light activated CRISPR-Cas9 system or the modulating CRISPR-Cas9 system (encapsulated/caged in the nanoshell) can be utilized to CRISPR-Cas9 mediated modification of a T-cell/CAR T-cell/CAR natural killer cell.

The T-cell/CAR T-cell/CAR natural killer cell can include or bind/couple (e.g., chemically bind/couple) with a sensing protein and/or an activating protein to detect a molecular event within a particular type of cancer cells.

The T-cell/CAR T-cell/CAR natural killer cell can include or bind/couple (e.g., chemically bind/couple) with the Ly49 protein. The Ly49 protein can bind with a Major Histocompatibility Complex (MHC) molecule to distinguish between a healthy cell and a particular type of cancer cells.

The T-cell/CAR T-cell/CAR natural killer cell can include or bind/couple (e.g., chemically bind/couple) with the Ly49 protein and/or a patient-specific Major Histocompatibility Complex protein.

The T-cell/CAR T-cell/CAR natural killer cell can include or bind/couple (e.g., chemically bind/couple) with the Ly49 protein and/or a synthetic Major Histocompatibility Complex molecule, wherein the synthetic Major Histocompatibility Complex molecule is further chemically coupled with a patient-specific peptide of a particular type of cancer cells.

The T-cell/CAR T-cell/CAR natural killer cell can include one or more deoxyribonucleic acid sequences to encode one or more therapeutic proteins against a particular type of cancer cells.

The T-cell/CAR T-cell/CAR natural killer cell can include one or more XNA sequences, wherein the one XNA sequence at least consists of one or more synthetic/artificial genetic bases (e.g., α and β) to encode one or more therapeutic proteins against a particular type of cancer cells.

The T-cell/CAR T-cell/CAR natural killer cell can include one or more (e.g., a first/second/third/fourth) nanoshells.

At least a first nanoshell can encapsulate one or more deoxyribonucleic acid sequences and/or one or more XNA sequences.

A second nanoshell can encapsulate one or more molecules (e.g., a synthetic p53 protein(s)/synthetic version of a cannabis flavonoid(s)) and/or an RNAi molecule(s) and/or modified (e.g., an overexpression of certain microRNAs and/or modified ligand(s)) exosomes beneficial against a particular type of cancer cells.

A third nanoshell can encapsulate a messenger RNA (mRNA) for coding Cas9 and a single-guide RNA (sgRNA). The single-guide RNA can scan the genome to help the nuclease find that specific sequence to be edited to fix mutation against a particular type of cancer cells.

The first/second/third nanoshell can be made of synthetic lipids comprising disulfide bonds in the fatty chain.

When the first/second/third nanoshell enters a cancer cell, the environment within the cancer cell breaks open the disulfide bond to disassemble the first/second/third nanoshell, subsequently the contents with specific purposes of the first/second/third nanoshell are quickly and efficiently released into the cell.

The first/second/third nanoshell can be coupled/chemically coupled with a molecular complex (which is encapsulated within human serum albumin), wherein the molecular complex includes a long-chain fatty acid integrated with a binding site for a molecule.

The first/second/third nanoshell can be a carbon nanodot.

A fourth nanoshell can be a liposome. The liposome can encapsulate bioactive molecules/drugs (beneficial against a particular type of cancer cells) and magnetic nanoparticles. Upon heating by a high frequency and low intensity magnetic field, the liposome can undergo a phase change from solid to liquid—thus releasing the bioactive molecules/drugs at a time t=0. But, when the high frequency and low intensity magnetic field is turned off, the lipids re-solidify due to reverse phase change, preventing any release of the bioactive molecules/drugs at a time t=t.

The carbon nanodot can be coupled/chemically coupled with a molecular complex (which is encapsulated within human serum albumin), wherein the molecular complex includes a long-chain fatty acid integrated with a binding site for a molecule.

The T-cell/CAR T-cell/CAR natural killer cell can include or bind/couple (e.g., chemically bind/couple) with one or more molecules/RNAi molecules/modified (e.g., an overexpression of certain microRNAs and/or modified ligand(s)) exosomes beneficial against a particular type of cancer cells.

Alternatively, the T-cell/CAR T-cell/CAR natural killer cell can include or bind/couple (e.g., chemically bind/couple) with a sensing protein and/or an activating protein to detect a molecular event within a particular type of cancer cells and a nanoshell encapsulating one or more deoxyribonucleic acid sequences and/or one or more XNA sequences and/or one or more with one or more molecules/RNAi molecules/modified (e.g., an overexpression of certain microRNAs and/or modified ligand(s)) exosomes beneficial against a particular type of cancer cells.

The nanoshell can be decorated with one or more targeting ligands to recognize/match/bind with specific biological receptors on a particular type of cancer cells.

The nanoshell can also include RNAi based logic circuit for computation regarding receptor based binding on a particular type of cancer cells.

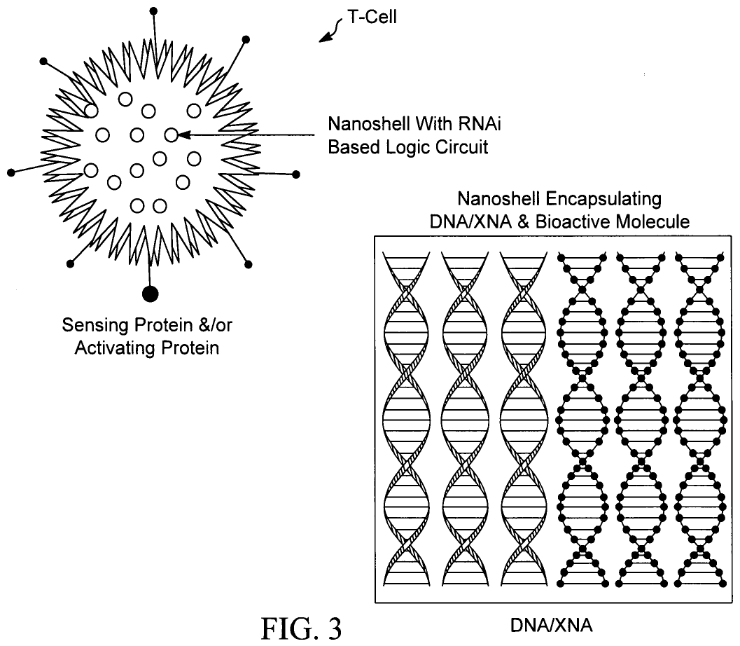

The T-cell with above configuration is illustrated in FIG. 3.

### Augmented & Engineered T-cell/Natural Killer Cell/CAR T-cell/CAR Natural Killer Cell

Furthermore the T-cell/CAR T-cell/CAR natural killer cell can include or bind/couple (e.g., chemically bind/couple) with deoxyribonucleic acid origami based nanostructure/ribonucleic acid (RNA) origami based nanostructure/XNA origami based nanostructure for recognition of a particular type of cancer cells.

The nanoshell (as described in previous paragraphs) can be coupled with a separate nanosystem.

The above separate nanosystem can be coated with lipid oleate and DOTAP to enhance its interaction and penetration into a particular type of cancer cells.

The separate nanosystem can include a tube shaped nanoscaled deoxyribonucleic acid cargo/carbon nanodot/nanoscaled metal (e.g., gold) rod (which is about 35 nanometers long and about 10 nanometers in radius).

The carbon nanodot is a fluorescent nanoparticle made of sucrose and citric acid and it is less toxic.

The tube shaped nanoscaled deoxyribonucleic acid cargo/carbon nanodot/nanoscaled metal rod can be coupled/chemically coupled with a molecular complex (which is encapsulated within human serum albumin (HSA)), wherein the molecular complex includes a long-chain fatty acid integrated with one or more binding sites for one or more molecules.

The tube shaped nanoscaled deoxyribonucleic acid cargo/carbon nanodot/nanoscaled metal rod can be coupled/chemically coupled with a heat shock protein and/or TRAIL protein.

Furthermore, the tube shaped nanoscaled deoxyribonucleic acid cargo/nanoscaled metal rod can include or couple (e.g., chemically couple) with a biocompatible metal (e.g., gold) nanoparticle, a nanoshell X and a nanoshell Y by strands of a biological material (e.g., apatmers/deoxyribonucleic acids/ribonucleic acids/XNAs).

The biocompatible metal nanoparticle, the nanoshell X and the nanoshell Y includes an immune shielding surface.

About fifteen (15) X/Y nanoshells can be coupled.

The strands of the biological material can include or couple (e.g., chemically couple) with one or more molecules beneficial against a particular type of cancer cells (e.g., a synthetic p53 protein(s)/synthetic version of a cannabis flavonoid(s)) and/or an RNAi molecule(s) and/or a photosensitizer molecule(s).

The nanoshell X can include or couple (e.g., chemically couple) with a near-infrared fluorescent polymer to visualize its accumulation at the target location of a particular type of cancer cells.

The nanoshell Y can be an upconverting nanoshell which converts a (continuous wave/pulsed) laser light of near-infrared wavelength into a (continuous wave/pulsed) laser light of visible wavelength.

The separate nanosystem can include one or more liposomes. Each liposome can encapsulate bioactive molecules/drugs and magnetic nanoparticles. Upon heating by a high frequency and low intensity magnetic field, the liposome can undergo a phase change from solid to liquid—thus releasing the bioactive molecules/drugs at a time t=0. But, when the high frequency and low intensity magnetic field is turned off, the lipids re-solidify due to reverse phase change, preventing any release of the bioactive molecules/drugs at a time t=t.

The separate nanosystem can further include or couple (e.g., chemically couple) with cerium fluoride nanoparticles and/or hafnium oxide.

Cerium fluoride nanoparticles can release reactive oxygen species upon activated/stimulated by X-rays of a (suitable) dose to destroy a particular type of cancer cells.

Hafnium oxide can generate electrons when exposed to X-rays of a (suitable) dose to destroy a particular type of cancer cells.

The nanoshell can include or bind/couple (e.g., chemically bind/couple) with biocompatible magnetic nanoparticles (e.g., Zn—Co—Cr-Ferrite nanoparticles or mn-co-Ferrite nanoparticles). Such biocompatible magnetic nanoparticles can be heated by a low magnetic field.

Combining the above approach with photodynamic therapy (PDT) via an optical fiber(s) can enhance therapeutic outcome against a particular type of cancer cells.

### Nanostructure Assisted Preferential Binding

A biologic pro-drug can be a non-toxic immune-stimulating drug (e.g., a non-toxic interleukin-2, when it binds only on beta and gamma units of a lymphocyte's surface).

Such preferential binding only on beta and gamma units of a lymphocyte's surface can be enabled by a combination of (a) polyethylene glycol (PEG) molecules and/or (b) a deoxyribonucleic acid origami based nanostructure/ribonucleic acid origami based nanostructure/XNA origami based nanostructure.

### Modulating CRISPR-Cas9 System

The modulating CRISPR-Cas9 system can activate an expression of one or more genes beneficial against a particular type of cancer cells.

The modulating CRISPR-Cas9 system can be encapsulated/caged in the nanoshell, wherein the nanoshell includes an immune shielding surface.

The nanoshell (encapsulating/caging the modulating CRISPR-Cas9 system) can include or bind/couple (e.g., chemically bind/couple) with a sensing protein and/or an activating protein to detect a molecular event within a particular type of cancer cells.

The nanoshell (encapsulating/caging the modulating CRISPR-Cas9 system) can include or bind/couple (e.g., chemically bind/couple) can include or bind/couple (e.g., chemically bind/couple) with one or more molecules/RNAi molecules/modified (e.g., an overexpression of certain microRNAs and/or modified ligand(s)) exosomes beneficial against a particular type of cancer cells.

### In Vivo Implanted, Biodegradable & Biocompatible (Artificial) Lymph Node

An implanted biodegradable and biocompatible (artificial) lymph node can be fabricated/constructed (e.g., a deoxyribonucleic acid origami based nanostructure/ribonucleic acid origami based nanostructure/XNA origami based nanostructure or a scaffold (e.g., three-dimensional) printed with a biocompatible and biodegradable polymer or a scaffold of silk fibroin) or a metallic glass material and then antibiotics can be added by soaking the scaffold in an antibiotic solution. Furthermore, the metallic glass material can be fabricated/constructed to have nanoscaled porosity and/or nanoscaled surface pattern for cell adhesion and/or inflammation.

The implanted biodegradable and biocompatible (artificial) lymph node can be integrated with a mechanically actuating membrane (which changes its shape in microscale level) to reduce unwanted cell growth near the implanted biodegradable and biocompatible (artificial) lymph node.

Alternatively, the above scaffold can be coated with a lattice of microgels and/or the whole assembly can be soaked/dipped at least two (2) times in an antibiotic solution).

The above scaffold can contain the Ly49 protein and/or a patient-specific Major Histocompatibility Complex protein.

The above scaffold can contain the Ly49 protein and/or a synthetic Major Histocompatibility Complex molecule, wherein the synthetic Major Histocompatibility Complex molecule is further chemically coupled with a patient-specific peptide of a particular type of cancer cells.

The above scaffold can contain one or more molecules (e.g., recruiting molecules and/or co-stimulating molecules (e.g., cytokines) and/or mobility enhancing molecules and/or programming molecules) and/or proteins on the surface of a particular type of cancer cells to recruit, attract and activate nearby immature dendritic cells at the site of the implanted biodegradable and biocompatible (artificial) lymph node.

The deoxyribonucleic acid origami based nanostructure/ribonucleic acid origami based nanostructure/XNA origami based nanostructure can be three-dimensional in shape.

The deoxyribonucleic acid origami based nanostructure/ribonucleic acid origami based nanostructure/XNA origami based nanostructure can be functionalized by modifying the oligonucleotide structures.

The deoxyribonucleic acid origami based nanostructure/ribonucleic acid origami based nanostructure/XNA origami based nanostructure can include RNAi based logic circuit regarding receptor computation on a particular type of cancer cells.

The inside and out side of the deoxyribonucleic acid origami based nanostructure/ribonucleic acid origami based nanostructure/XNA origami based nanostructure can be functionalized by a precise spatial pattern of ligands to induce/activate dendritic cells in order to initiate response of the T-cells and/or natural killer cells.

To overcome the immunosurveillance, the deoxyribonucleic acid origami based nanostructure/ribonucleic acid origami based nanostructure/XNA origami based nanostructure can be attached with a red blood cell/white blood cell.

Alternatively, to overcome the immunosurveillance, the deoxyribonucleic acid origami based nanostructure/ribonucleic acid origami based nanostructure/XNA origami based nanostructure can be encapsulated in a lipid bilayer.

The engineered dendritic cell exchanges or transfers the sensing protein to the T-cell, and/or the natural killer cell.

The engineered dendritic cell can include one or more (e.g., a first/second/third/fourth) nanoshells.

A first nanoshell can encapsulate one or more deoxyribonucleic acid sequences and/or one or more XNA sequences.

A second nanoshell can encapsulate one or more molecules (e.g., a synthetic p53 protein(s)/synthetic version of a cannabis flavonoid(s)) and/or an RNAi molecule(s) and/or modified (e.g., an overexpression of certain microRNAs and/or modified ligand(s)) exosomes beneficial against a particular type of cancer cells.

A third nanoshell can encapsulate a messenger RNA (mRNA) for coding Cas9 and a single-guide RNA (sgRNA). The single-guide RNA can scan the genome to help the nuclease find that specific sequence to be edited to fix mutation against a particular type of cancer cells.

For example, a cannabis flavonoid is any of these molecules: Cannflavin A, Cannflavin B, Cannflavin C, Chrysoeriol, Cosmosiin and Flavocannabiside—but not limited to these examples.

The first nanoshell or the second nanoshell or the third nanoshell can be coupled/chemically coupled with a molecular complex (which is encapsulated within human serum albumin), wherein the molecular complex includes a long-chain fatty acid integrated with one or more binding sites for one or more molecules.

The first nanoshell or the second nanoshell or the third nanoshell can be a carbon nanodot.

The engineered dendritic cell can exchange or transfer the first/second/third nanoshell to the T-cell and/or the natural killer cell.

At least a first nanoshell can encapsulate one or more deoxyribonucleic acid sequences and/or one or more XNA sequences.

A second nanoshell can encapsulate one or more molecules (e.g., a synthetic p53 protein(s)/synthetic version of a cannabis flavonoid(s)) and/or an RNAi molecule(s) and/or modified (e.g., an overexpression of certain microRNAs and/or modified ligand(s)) exosomes beneficial against a particular type of cancer cells.

A third nanoshell can encapsulate a messenger RNA (mRNA) for coding Cas9 and a single-guide RNA (sgRNA). The single-guide RNA can scan the genome to help the nuclease find that specific sequence to be edited to fix mutation against a particular type of cancer cells.

A fourth nanoshell can be a liposome. The liposome can encapsulate bioactive molecules/drugs (beneficial against a particular type of cancer cells) and magnetic nanoparticles. Upon heating by a high frequency and low intensity magnetic field, the liposome can undergo a phase change from solid to liquid—thus releasing the bioactive molecules/drugs at a time t=0. But, when the high frequency and low intensity magnetic field is turned off, the lipids re-solidify due to reverse phase change, preventing any release of the bioactive molecules/drugs at a time t=t.

### CRISPR-Cas9 System/Light Activated CRISPR-Cas9 System

These immature dendritic cells can include the CRISPR-Cas9 system or the light activated CRISPR-Cas9 system.

The CRISPR-Cas9 system or the light activated CRISPR-Cas9 system can be encapsulated/caged in the nanoshell, wherein the nanoshell includes an immune shielding, wherein the CRISPR-Cas9 system or the light activated CRISPR-Cas9 system can remove a first gene responsible for inactivation of immature dendritic cells and add/insert/replace a second gene (including genetic bases A (adenine), C (cytosine), T (thymine), G (guanine) and/or one or more synthetic/artificial genetic bases (e.g., α and β)) to express/release a molecule (e.g., an antibody/hormone/protein) for enhanced interaction of dendritic cells with the T-cells and/or natural killer cells.

Inserting genes in the point cleaved by CRISPR-Cas9 system may require other proteins. A cell may contain or can be configured to contain such proteins. Thus, a complementary sequence on the ends of the insertion sequence may be needed to add/insert/replace a second gene (including genetic bases A (adenine), C (cytosine), T (thymine), G (guanine) and/or one or more synthetic/artificial genetic bases (e.g., α and β)) inside a cell.

The nanoshell (encapsulating/caging the CRISPR-Cas9 system or the light activated CRISPR-Cas9 system) can include or bind/couple (e.g., chemically bind/couple) with a sensing protein and/or an activating protein to detect a molecular event within a particular type of cancer cells.

The nanoshell (encapsulating/caging the CRISPR-Cas9 system or the light activated CRISPR-Cas9 system) can include or bind/couple (e.g., chemically bind/couple) with one or more molecules/RNAi molecules/modified (e.g., an overexpression of certain microRNA(s) and/or modified ligand(s)) exosomes beneficial against a particular type of cancer cells.

### Modulating CRISPR-Cas9 System

These immature dendritic cells can include the modulating CRISPR-Cas9 system.

The modulating CRISPR-Cas9 system can activate an expression of one or more genes beneficial against a particular type of cancer cells.

The modulating CRISPR-Cas9 system can be encapsulated/caged in the nanoshell, wherein the nanoshell includes an immune shielding surface.

The nanoshell (encapsulating/caging the modulating CRISPR-Cas9 system) can include or bind/couple (e.g., chemically bind/couple) with a sensing protein and/or an activating protein to detect a molecular event within a particular type of cancer cells.

The nanoshell (encapsulating/caging the modulating CRISPR-Cas9 system) can include or bind/couple (e.g., chemically bind/couple) can include or bind/couple (e.g., chemically bind/couple) with one or more molecules/RNAi molecules/modified (e.g., an overexpression of certain microRNAs and/or modified ligand(s)) exosomes beneficial against a particular type of cancer cells.

These activated dendritic cells can then travel to nearby natural lymph nodes, where these can train other types of immune cells (especially the T-cells and/or natural killer cells) to recognize and destroy existing cancer cells in the human body, if any and/or protect against future recurrence of any cancer cells, elsewhere in the human body.

In general, but not limited to, an engineered dendritic cell (in vivo), wherein the engineered dendritic cell is residing in a biocompatible/biodegradable lymph node, wherein the lymph node is coated with a lattice of microgels or dipped in an antibiotic solution, wherein the engineered dendritic cell includes:
(a) a first molecule,
wherein the first molecule is selected from the group consisting of the following: a co-stimulating molecule, a mobility enhancing molecule and a programming molecule,
(b) an identifying protein on the surface of a particular type of cancer cells, or
a sensing protein to detect a particular type of cancer cells,
(c) a nanostructure or a scaffold of biocompatible polymers or a scaffold of silk-fibroin, wherein the nanostructure includes a deoxyribonucleic acid origami based nanostructure or a ribonucleic acid origami based nanostructure or a XNA origami based nanostructure for interaction with the T-cells and/or the natural killer cells against a particular type of cancer cells, wherein XNA includes genetic bases adenine (A), thymine (T), guanine (G), cytosine (C) and uracil (U) and one or more synthetic or artificial genetic bases, wherein the nanostructure is functionalized by a pattern of ligands to induce or activate dendritic cell to initiate response of the T-cells and/or the natural killer cells, wherein the nanostructure is encapsulated in a lipid bilayer or the nanostructure is attached with a red blood cell or a white blood cell.

The engineered dendritic cell in above, can contain the Ly49 protein and/or a patient-specific Major Histocompatibility Complex protein.

The engineered dendritic cell in above, can contain the Ly49 protein and/or a synthetic Major Histocompatibility Complex molecule, wherein the synthetic Major Histocompatibility Complex molecule is further chemically coupled with a patient-specific peptide of a particular type of cancer cells.

The engineered dendritic cell in above, further exchanges or transfers the sensing protein to the T-cells and/or the natural killer cells.

The engineered dendritic cell in above, further includes a nanoshell, wherein the nanoshell encapsulates deoxyribonucleic acid sequence and/or XNA sequence to encode a therapeutic protein against a particular type of cancer cells, wherein XNA includes genetic bases adenine (A), thymine (T), guanine (G), cytosine (C) and uracil (U) and one or more synthetic or artificial genetic bases.

The engineered dendritic cell in above, further exchanges or transfers the nanoshell with to the T-cells and/or the natural killer cells, wherein the nanoshell releases deoxyribonucleic acid sequence and/or XNA sequence to encode a therapeutic protein against a particular type of cancer cells within the T-cells and/or the natural killer cells, wherein XNA includes genetic bases adenine (A), thymine (T), guanine (G), cytosine (C) and uracil (U) and one or more synthetic or artificial genetic bases.

The engineered dendritic cell in above, wherein the nanostructure includes RNAi based logic circuit.

The engineered dendritic cell in above, further includes a CRISPR-Cas9 system or a light activated CRISPR-Cas9 system, wherein the CRISPR-Cas9 system or the light activated CRISPR-Cas9 system removes a first defective gene for inactivation of a dendritic cell and adds/inserts/replaces a second gene, wherein the added/inserted/replaced second gene expresses, or releases a third molecule to increase interaction with the T-cells and/or the natural killer cells. The added/inserted/replaced second gene includes one or more synthetic or artificial genetic bases.

The Cas9 can be replaced by Cas9-HFI or Cas12a. CRISPR-Cas9 system can be replaced by a transposon-encoded CRISPR-Cas system.

The engineered dendritic cell in above, includes a modulating CRISPR-Cas9 system, wherein the modulating CRISPR-Cas9 system consists of Cas9, a short single guide RNA, a transcriptional activator and an adeno-associated virus (AAV), wherein the modulating CRISPR-Cas9 system activates an expression of one or more genes beneficial gene against a particular type of cancer cells.

### Ex Vivo Engineered/Edited Dendritic Cells

Instead of an implanted biodegradable and biocompatible (artificial) lymph node, the activation of the dendritic cells by one or more molecules (e.g., co-stimulating molecules (e.g., cytokines) and/or mobility enhancing molecules and/or programming molecules) and/or proteins on the surface of a particular type of cancer cells and/or sensing proteins to detect a particular type of cancer cells can be realized in ex vivo

The engineered dendritic cell (about 7 microns in diameter) can include or bind/couple (e.g., chemically bind/couple) with the Ly49 protein and/or a patient-specific Major Histocompatibility Complex protein.

The engineered dendritic cell can include or bind/couple (e.g., chemically bind/couple) with the Ly49 protein and/or a synthetic Major Histocompatibility Complex molecule, wherein the synthetic Major Histocompatibility Complex molecule is further chemically coupled with a patient-specific peptide of a particular type of cancer cells.

The engineered dendritic cell can include or bind/couple (e.g., chemically bind/couple) with the deoxyribonucleic acid origami based nanostructure/ribonucleic acid origami based nanostructure/XNA origami based nanostructure for enhanced interaction with the T-cells and/or natural killer cells.

The deoxyribonucleic acid origami based nanostructure/ribonucleic acid origami based nanostructure/XNA origami based nanostructure can be three-dimensional in shape.

To overcome the immunosurveillance, the deoxyribonucleic acid origami based nanostructure/ribonucleic acid origami based nanostructure/XNA origami based nanostructure can be attached with a red blood cell/white blood cell.

Alternatively, to overcome the immunosurveillance, the deoxyribonucleic acid origami based nanostructure/ribonucleic acid origami based nanostructure/XNA origami based nanostructure can be encapsulated in a lipid bilayer.

Furthermore, the deoxyribonucleic acid origami based nanostructure/ribonucleic acid origami based nanostructure/XNA origami based nanostructure can include or couple (e.g., chemically couple) with RNAi based logic circuit for precise/enhanced interaction of the engineered dendritic cells with the T-cells and/or natural killer cells.

RNAi based logic circuit can be capable of deciphering (computation by RNAi-based logic circuit) the cellular transcriptome, which calculates levels of targeted biomarkers and determines whether cells are either healthy or cancerous by evaluating internal cell state through mRNA expression patterns specifically via an overexpression of Gata3, NPYIR and TFFI (as these are generally present in cancer cells).

The engineered dendritic cell can include one or more (e.g., a first/second/third/fourth) nanoshells.

At least a first nanoshell can encapsulate one or more deoxyribonucleic acid sequences and/or one or more XNA sequences.

A second nanoshell can encapsulate one or more molecules (e.g., a synthetic p53 protein(s)/synthetic version of a cannabis flavonoid(s)) and/or an RNAi molecule(s) and/or modified (e.g., an overexpression of certain microRNAs and/or modified ligand(s)) exosomes beneficial against a particular type of cancer cells.

A third nanoshell can encapsulate a messenger RNA (mRNA) for coding Cas9 and a single-guide RNA (sgRNA). The single-guide RNA can scan the genome to help the nuclease find that specific sequence to be edited to fix mutation against a particular type of cancer cells.

The first nanoshell or the second nanoshell or the third nanoshell can be coupled/chemically coupled with a molecular complex (which is encapsulated within human serum albumin), wherein the molecular complex includes a long-chain fatty acid integrated with one or more binding sites for one or more molecules.

The first nanoshell or the second nanoshell or the third nanoshell can be a carbon nanodot.

A fourth nanoshell can be a liposome. The liposome can encapsulate bioactive molecules/drugs (beneficial against a particular type of cancer cells) and magnetic nanoparticles. Upon heating by a high frequency and low intensity magnetic field, the liposome can undergo a phase change from solid to liquid—thus releasing the bioactive molecules/drugs at a time t=0. But, when the high frequency and low intensity magnetic field is turned off, the lipids re-solidify due to reverse phase change, preventing any release of the bioactive molecules/drugs at a time t=t.

When injected into human body, the engineered dendritic cell may then travel to nearby natural lymph nodes, where these can train another immune cell (especially the T-cells and/or natural killer cells) to recognize and destroy existing cancer cells in the human body, if any and/or protect against future recurrence of any cancer cells, elsewhere in the human body.

When injected into human body, the engineered dendritic cell may then travel to nearby natural lymph nodes, where these can exchange with another immune cell (especially the T-cells and/or natural killer cells) the sensing protein to recognize and destroy existing cancer cells in the human body, if any and/or protect against future recurrence of any cancer cells, elsewhere in the human body.

Moreover, when injected into human body, the engineered dendritic cell may then travel to nearby natural lymph nodes, where these can transfer with another immune cell (especially the T-cells and/or natural killer cells) the nanoshell. Upon transfer with another immune cell via eceptor-mediated endocytosis, the nanoshell can release one or more deoxyribonucleic acid sequences to encode one or more therapeutic proteins against a particular type of cancer cells.

Moreover, when injected into human body, the engineered dendritic cell may then travel to nearby natural lymph nodes, where these can transfer with another immune cell (especially the T-cells and/or natural killer cells) the nanoshell. Upon transfer with another immune cell via eceptor-mediated endocytosis, the nanoshell can release one or more XNA sequences to encode one or more therapeutic proteins against a particular type of cancer cells.

### Ex Vivo Engineered/Edited Dendritic Cells, Utilizing CRISPR-Cas9 System/Light Activated CRISPR-Cas9 System

These dendritic cells can include the CRISPR-Cas9 system or the light activated CRISPR-Cas9 system. Thus, these are engineered/edited dendritic cells.

The CRISPR-Cas9 system or the light activated CRISPR-Cas9 system can be encapsulated/caged in the nanoshell, wherein the nanoshell includes an immune shielding, wherein the CRISPR-Cas9 system or the light activated CRISPR-Cas9 system can remove a first gene responsible for inactivation of immature dendritic cells and add/insert/replace a second gene (including genetic bases A (adenine), C (cytosine), T (thymine), G (guanine) and/or one or more synthetic/artificial genetic bases (e.g., α and β)) to express/release a molecule (e.g., an antibody/hormone/protein) for enhanced interaction of the dendritic cell with the T-cell and/or natural killer cell.

Autophagy is a process of maintaining cell homeostasis by removing cellular waste and damaged cellular organelles. Autophagy of the dendritic cell can activate the T-cell's anticancer activity. Furthermore, autophagy related Atg5-deficient dendritic cell has significantly elevated receptor CD36 on its surface, which increased the phagocytosis of apoptotic cancer cell, but restricted the activation of the T-cell. Thus, controlling receptor CD36 on the surface of the dendritic cell by the CRISPR-Cas9 system or the light activated CRISPR-Cas9 system can activate the T-cell's anticancer activity.

Inserting genes in the point cleaved by CRISPR-Cas9 system may require other proteins. A cell may contain or can be configured to contain such proteins. Thus, a complementary sequence on the ends of the insertion sequence may be needed to add/insert/replace a second gene (including genetic bases A (adenine), C (cytosine), T (thymine), G (guanine) and/or one or more synthetic/artificial genetic bases (e.g., α and β)) inside a cell.

The nanoshell (encapsulating/caging the CRISPR-Cas9 system or the light activated CRISPR-Cas9 system) can include or bind/couple (e.g., chemically bind/couple) with a sensing protein and/or an activating protein to detect a molecular event within a particular type of cancer cells.

The nanoshell can include one or more deoxyribonucleic acid/XNA sequences to encode one or more therapeutic proteins.

The nanoshell (encapsulating/caging the CRISPR-Cas9 system or the light activated CRISPR-Cas9 system) can include or bind/couple (e.g., chemically bind/couple) with one or more molecules/RNAi molecules/modified (e.g., an overexpression of certain microRNAs and/or modified ligand(s)) exosomes beneficial against a particular type of cancer cells.

### Ex Vivo Engineered/Edited Dendritic Cells, Utilizing Modulating CRISPR-Cas9 System

These dendritic cells can include the modulated CRISPR-Cas9 system. Thus, these are engineered/edited dendritic cells.

The modulating CRISPR-Cas9 system can activate an expression of one or more genes beneficial against a particular type of cancer cells.

The modulating CRISPR-Cas9 system can be encapsulated/caged in the nanoshell, wherein the nanoshell includes an immune shielding surface.

The nanoshell (encapsulating/caging the modulating CRISPR-Cas9 system) can include or bind/couple (e.g., chemically bind/couple) with a sensing protein and/or an activating protein to detect a molecular event within a particular type of cancer cells.

The nanoshell can include one or more deoxyribonucleic acid/XNA sequences to encode one or more therapeutic proteins.

Autophagy is a process of maintaining cell homeostasis by removing cellular waste and damaged cellular organelles. Autophagy of the dendritic cell can activate the T-cell's anticancer activity. Furthermore, autophagy related Atg5-deficient dendritic cell has significantly elevated receptor CD36 on its surface, which increased the phagocytosis of apoptotic cancer cell, but restricted the activation of the T-cell. Thus, controlling receptor CD36 on the surface of the dendritic cell by the modulating CRISPR-Cas9 system can activate the T-cell's anticancer activity.

The nanoshell (encapsulating/caging the modulating CRISPR-Cas9 system) can include or bind/couple (e.g., chemically bind/couple) can include or bind/couple (e.g., chemically bind/couple) with one or more molecules/RNAi molecules/modified (e.g., an overexpression of certain microRNAs and/or modified ligand(s)) exosomes beneficial against a particular type of cancer cells

When injected into human body, these engineered/edited dendritic cells may then travel to nearby natural lymph nodes, where these can train other types of immune cells (especially the T-cells and/or natural killer cells) to recognize and destroy existing cancer cells in the human body, if any and/or protect against future recurrence of any cancer cells, elsewhere in the human body.

The engineered dendritic cell can contain the nanoshell, wherein one or more deoxyribonucleic acid sequences to encode one or more therapeutic proteins against a particular type of cancer cells are encapsulated within the nanoshell.

Furthermore, the engineered dendritic cell can contain the nanoshell, wherein one or more XNA sequences to encode one or more therapeutic proteins against a particular type of cancer cells are encapsulated within the nanoshell.

When injected into human body, the engineered dendritic cell may then travel to nearby natural lymph nodes, where these can train another immune cell (especially the T-cells and/or natural killer cells) to recognize and destroy existing cancer cells in the human body, if any and/or protect against future recurrence of any cancer cells, elsewhere in the human body.

When injected into human body, the engineered dendritic cell may then travel to nearby natural lymph nodes, where these can exchange with another immune cell (especially the T-cells and/or natural killer cells) the sensing protein to recognize and destroy existing cancer cells in the human body, if any and/or protect against future recurrence of any cancer cells, elsewhere in the human body.

Moreover, when injected into human body, the engineered dendritic cell may then travel to nearby natural lymph nodes, where these can transfer with another immune cell (especially the T-cells and/or natural killer cells) the nanoshell. Upon transfer with another immune cell via eceptor-mediated endocytosis, the nanoshell can release one or more deoxyribonucleic acid sequences to encode one or more therapeutic proteins against a particular type of cancer cells.

Moreover, when injected into human body, the engineered dendritic cell may then travel to nearby natural lymph nodes, where these can transfer with another immune cell (especially the T-cells and/or natural killer cells) the nanoshell. Upon transfer with another immune cell via eceptor-mediated endocytosis, the nanoshell can release one or more XNA sequences to encode one or more therapeutic proteins against a particular type of cancer cells.

Additionally, an engineered T-cells/natural killer cells can include or bind/couple (e.g., chemically bind/couple) with deoxyribonucleic acid origami nanostructure/ribonucleic acid origami nanostructure/XNA origami nanostructure for enhanced interaction with the engineered/edited dendritic cells.

Additionally, the engineered T-cells/natural killer cells can include one or more deoxyribonucleic acid/XNA sequences to encode one or more therapeutic proteins.

In general, but not limited to, an engineered dendritic cell (ex vivo) includes:
(a) a first molecule,
wherein the first molecule is selected from the group consisting of the following: a co-stimulating molecule, a mobility enhancing molecule and a programming molecule,
(b) an identifying protein on the surface of a particular type of cancer cells, or
a sensing protein to detect a particular type of cancer cells and
(c) a nanostructure,
wherein the nanostructure includes a deoxyribonucleic acid origami based nanostructure or a ribonucleic acid origami based nanostructure or a XNA origami based nanostructure for interaction with the T-cells and/or the natural killer cells against a particular type of cancer cells, wherein XNA includes genetic bases adenine (A), thymine (T), guanine (G), cytosine (C) and uracil (U) and one or more synthetic or artificial genetic bases, wherein the nanostructure is encapsulated in a lipid bilayer, or the nanostructure is attached with a red blood cell, or a white blood cell.

The engineered dendritic cell in above, further includes a second molecule to initiate response of the T-cells and/or the natural killer cells.

The engineered dendritic cell in above, further includes an activating protein to detect a molecular event within a particular type of cancer cells.

The engineered dendritic cell in above, further exchanges or transfers the sensing protein to the T-cells and/or the natural killer cells.

The engineered dendritic cell in above, further includes or binds/couples (e.g., chemically bind/couple) with the Ly49 protein and/or a patient-specific Major Histocompatibility Complex protein.

The engineered dendritic cell in above, further includes or binds/couples (e.g., chemically bind/couple) with the Ly49 protein and/or a synthetic Major Histocompatibility Complex molecule, wherein the synthetic Major Histocompatibility Complex molecule is further chemically coupled with a patient-specific peptide of a particular type of cancer cells.

The engineered dendritic cell in above, further includes a nanoshell, wherein the nanoshell encapsulates deoxyribonucleic acid sequence and/or XNA sequence to encode a therapeutic protein against a particular type of cancer cells, wherein XNA includes genetic bases adenine (A), thymine (T), guanine (G), cytosine (C) and uracil (U) and one or more synthetic or artificial genetic bases.

The engineered dendritic cell in above, further exchanges or transfers the nanoshell to the T-cells and/or the natural killer cells, wherein the nanoshell releases deoxyribonucleic acid sequence and/or XNA sequence to encode a therapeutic protein against a particular type of cancer cells within the T-cells and/or the natural killer cells, wherein XNA includes genetic bases adenine (A), thymine (T), guanine (G), cytosine (C) and uracil (U) and one or more synthetic or artificial genetic bases.

The engineered dendritic cell in above, wherein the nanostructure further includes RNAi based logic circuit.

The engineered dendritic cell in above, wherein the nanostructure is functionalized by a pattern of ligands to induce or activate dendritic cell to initiate response of the T-cells and/or the natural killer cells.

The engineered dendritic cell in above, further includes a CRISPR-Cas9 system or a light activated CRISPR-Cas9 system, wherein the CRISPR-Cas9 system or the light activated CRISPR-Cas9 system removes a first defective gene for inactivation of a dendritic cell, and adds/inserts/replaces a second gene, wherein the added/inserted/replaced second gene expresses, or releases a third molecule to increase interaction with the T-cells and/or the natural killer cells. The added/inserted/replaced second gene includes one or more synthetic or artificial genetic bases.

The Cas9 can be replaced by Cas9-HFI or Cas12a. CRISPR-Cas9 system can be replaced by a transposon-encoded CRISPR-Cas system.

The engineered dendritic cell in above, further couples with or includes one or more modified exosomes.

The engineered dendritic cell in above, is an engineered and modulated dendritic cell, wherein the engineered dendritic cell further includes a modulating CRISPR-Cas9 system, wherein the modulating CRISPR-Cas9 system includes Cas9, a short single guide RNA, a transcriptional activator and an adeno-associated virus, wherein the modulating CRISPR-Cas9 system activates an expression of one or more genes beneficial gene against a particular type of cancer cells.

### Application to Editing B Cells, Utilizing CRISPR-Cas9 System/Light Activated CRISPR-Cas9 System or Modulating CRISPR-Cas9 System to Create Specific Antibody

The CRISPR-Cas9 system/light activated CRISPR-Cas9 system or modulating CRISPR-Cas9 system can be utilized to modify B cells (a class of white blood cells) with genes that create specific antibodies that produce antibodies to protect from diseases and/or inflammation.

Furthermore, genes can include natural genetic bases such as adenine (A), thymine (T), guanine (G), cytosine (C) and uracil (U) and/or one or more synthetic/artificial genetic bases (e.g., α and β).

Furthermore, the CRISPR-Cas9 system/light activated CRISPR-Cas9 system or modulating CRISPR-Cas9 system can be encapsulated in the nanoshell.

Additionally, the nanoshell can include synthetically created a small chain(s) of amino acids or a polypeptide for triggering B cells to create specific antibodies.

As an example, but not limited to, a synthetic polypeptide for a specific beta amyloid protein (Alzheimer's disease) can be a formula Vab-Rn—X—P—Y—Rn-Vab, wherein the synthetic polypeptide is engineered and chemically conjugated, wherein each variable antigen-binding (Vab) domain includes a binding site for an antigen and all or a portion of the Vab domains are from camelid single-domain heavy chain antibodies lacking light-chains, wherein each Rn independently includes a camelid hinge region from a single domain heavy chain antibody lacking light chains, wherein X is a human immunoglobulin CH2 domain, wherein P is —(CH₂—CH₂—O)ₙ—, wherein n=4, wherein Y is a succinimidyl moiety, wherein Y further includes—S—(CH₂)₃—C(NH₂Cl)NH—, wherein the synthetic polypeptide binds or couples to a segment of a beta amyloid protein, wherein the beta amyloid protein has an amino acid sequence (Sequence ID No: 52): MDAEFRHDSG YEVHHQKLVF FAEDVGSNKG AIIGLMVGGV VIATVIVITL VMLKKKQYTS IHHGVVEVGK LDCMFPSGN, wherein the synthetic polypeptide has a molecular weight between 25 kDa and 90 kDa.

Similarly, a synthetic polypeptide for a specific tau protein (Alzheimer's disease) can be realized.

Application to Treatment of My with an Engineered/Edited or Modulated Stem Cell

In order for HIV, the virus causing AIDS, to enter cells it usually must fuse with a receptor called CCR5 that sits on the surface of T-helper immune cells.

The delta-32 mutation in the gene encoding the CCR5 protein results in a defective/mutated receptor site that can block the entry of the virus causing AIDS.

The CRISPR-Cas9 system or the light activated CRISPR-Cas9 system via the nanoshell can be inserted into a stem cell to remove a first gene (or first genes) that promote the development of mutation of the receptor called CCR5 and/or additionally, the CRISPR-Cas9 system or the light activated CRISPR-Cas9 system via the nanoshell can be inserted into a stem cell to add/insert/replace a second gene (or genes) that promote the development of mutation of the receptor called CCR5.

Thus, a mutation strategy of the receptor called CCR5 can block the entry of the virus causing AIDS.

### Application to Genetic Medicine, Utilizing CRISPR-Cas9 System/Light Activated CRISPR-Cas9 System

The nanoshell (encapsulating/caging the CRISPR-Cas9 system or the light activated CRISPR-Cas9 system) can include a drug printed by a three-dimensional printer/bio-ink printer.

The CRISPR-Cas9 system or the light activated CRISPR-Cas9 system can be encapsulated/caged in the nanoshell, wherein the nanoshell includes an immune shielding surface, wherein the CRISPR-Cas9 system or the light activated CRISPR-Cas9 system can remove a first gene responsible for a disease and add/insert/replace a second gene (including genetic bases A (adenine), C (cytosine), T (thymine), G (guanine) and/or one or more synthetic/artificial genetic bases (e.g., α and β)) to express/release a molecule (e.g., an antibody/hormone/protein), beneficial against a particular type of diseased cells.

Inserting genes in the point cleaved by CRISPR-Cas9 system may require other proteins. A cell may contain or can be configured to contain such proteins. Thus, a complementary sequence on the ends of the insertion sequence may be needed to add/insert/replace a second gene (including genetic bases A (adenine), C (cytosine), T (thymine), G (guanine) and/or one or more synthetic/artificial genetic bases (e.g., α and β)) inside a cell.

The nanoshell (encapsulating/caging the CRISPR-Cas9 system or the light activated CRISPR-Cas9 system) can include or bind/couple (e.g., chemically bind/couple) with a sensing protein and/or an activating protein to detect a molecular event within a particular type of diseased cells.

The nanoshell (encapsulating/caging the CRISPR-Cas9 system or the light activated CRISPR-Cas9 system) can include or bind/couple (e.g., chemically bind/couple) with one or more molecules/RNAi molecules/modified (e.g., an overexpression of certain microRNAs and/or modified ligand(s)) exosomes beneficial against a particular type of diseased cells

### Application to Genetic Medicine, Utilizing Modulating CRISPR-Cas9 System

The modulating CRISPR-Cas9 system can activate an expression of one or more genes beneficial genes against a particular type of diseased cells.

The modulating CRISPR-Cas9 system can be encapsulated/caged in the nanoshell, wherein the nanoshell includes an immune shielding surface.

The nanoshell (encapsulating/caging the modulating CRISPR-Cas9 system) can include or bind/couple (e.g., chemically bind/couple) with a sensing protein and/or an activating protein to detect a molecular event within a particular type of diseased cells.

The nanoshell (encapsulating/caging the modulating CRISPR-Cas9 system) can include or bind/couple (e.g., chemically bind/couple) can include or bind/couple (e.g., chemically bind/couple) with one or more molecules/RNAi molecules/modified (e.g., an overexpression of certain microRNAs and/or modified ligand(s)) exosomes beneficial against a particular type of diseased cells

Application to Transplantable Organ with Stem Cell, Utilizing CRISPR-Cas9 System/Light Activated CRISPR-Cas9 System or Modulating CRISPR-Cas9 System

A human induced pluripotent stem cells can be injected into the animal's embryo and placed into womb of the female animal (e.g., a pig/sheep) to grow a human organ—resulting in birth of an adult animal (e.g., a pig/sheep) with the human organ after nine (9) months.

Furthermore, the human induced pluripotent stem cells can be engineered/edited utilizing the CRISPR-Cas9 system or the light activated CRISPR-Cas9 system or the modulating CRISPR-Cas9 system.

Then, the human organ can be transplanted to a patient, upon slaughtering the adult animal with the human organ.

### Application to Stem Cell Derived Beta Cells, Utilizing CRISPR-Cas9 System/Light Activated CRISPR-Cas9 System or Modulating CRISPR-Cas9 System

Immature pancreatic cells (beta cells) can be derived from human embryonic stem cells. Such immature pancreatic cells (beta cells) can be edited utilizing CRISPR-Cas9 system or light activated CRISPR-Cas9 system, encapsulated in the nanoshell.

Alternatively, such immature pancreatic cells (beta cells) can be modulated utilizing modulating CRISPR-Cas9 system, encapsulated in the nanoshell.

The edited/modulated immature pancreatic cells (beta cells) can be encapsulated to protect from the human body's immune system. The encapsulated edited/modulated immature pancreatic cells (beta cells) can be implanted under the skin of the human body to manage insulin production for either Type-1 diabetes or Type-2 diabetes

### Replacement by Cas9-HFI or Cas12a

In all previous paragraphs, high-fidelity Cas9-HFI or a suitable molecule (e.g., Cas12a) can be utilized instead of Cas9 for reduced off-targets.

Replacement by Transposon-Encoded CRISPR-Cas System (with Jumping Genes)

A first concern with is that editing with CRISPR-Cas9 system can alter deoxyribonucleic acid in places it is not supposed to be there and these off-target may trigger cancers. A second concern is that a cell can insert a random deoxyribonucleic acid when it is making repairs with CRISPR-Cas9 system and these could silence needed genes. A third concern is that it is harder to insert the right new genes with CRISPR-Cas9 system. A transposon-encoded CRISPR-Cas system (with jumping genes) can be utilized instead of CRISPR-Cas9 system to reduce the above mentioned first, second and third concerns.

### Preferred Embodiments & Scope of the Invention

As used in the above disclosed specifications, the above disclosed specifications “/” has been used to indicate an “or”.

As used in the above disclosed specifications and in the claims, the singular forms “a”, “an”, and “the” include also the plural forms, unless the context clearly dictates otherwise.

As used in the above disclosed specifications, the term “includes” means “comprises”. Also the term “including” means “comprising”.

As used in the above disclosed specifications, the term “couples” or “coupled” does not exclude the presence of an intermediate element(s) between the coupled items.

As used in the above disclosed specifications, any weight % in the above disclosed specifications is by way of an approximation only and not by way of any limitation.

Any dimension in the above disclosed specifications is by way of an approximation only and not by way of any limitation.

As used in the above disclosed specifications, unless otherwise specified in the relevant paragraph(s), a nanoscaled dimension shall generally mean a dimension from about 1 nm to about 1000 nm.

Any example in the above disclosed specifications is by way of an example only and not by way of any limitation. Having described and illustrated the principles of the disclosed technology with reference to the illustrated embodiments, it will be recognized that the illustrated embodiments can be modified in any arrangement and detail with departing from such principles. The technologies from any example can be combined in any arrangement with the technologies described in any one or more of the other examples. Alternatives specifically addressed in this application are merely exemplary and do not constitute all possible examples. Claimed invention is disclosed as one of several possibilities or as useful separately or in various combinations. See Novozymes A/S v. DuPont Nutrition Biosciences APS, 723 F3d 1336,1347.

The best mode requirement “requires an inventor(s) to disclose the best mode contemplated by him/her, as of the time he/she executes the application, of carrying out the invention.” “ . . . [T]he existence of a best mode is a purely subjective matter depending upon what the inventor(s) actually believed at the time the application was filed.” See Bayer AG v. Schein Pharmaceuticals, Inc. The best mode requirement still exists under the America Invents Act (AIA). At the time of the invention, the inventor(s) described preferred best mode embodiments of the present invention. The sole purpose of the best mode requirement is to restrain the inventor(s) from applying for a patent, while at the same time concealing from the public preferred embodiments of their inventions, which they have in fact conceived. The best mode inquiry focuses on the inventor(s)′ state of mind at the time he/she filed the patent application, raising a subjective factual question. The specificity of disclosure required to comply with the best mode requirement must be determined by the knowledge of facts within the possession of the inventor(s) at the time of filing the patent application. See Glaxo, Inc. v. Novopharm Ltd., 52 F.3d 1043, 1050 (Fed. Cir. 1995). The above disclosed specifications are the preferred best mode embodiments of the present invention. However, they are not intended to be limited only to the preferred best mode embodiments of the present invention.

Embodiment by definition is a manner in which an invention can be made or used or practiced or expressed. “A tangible form or representation of the invention” is an embodiment.

Numerous variations and/or modifications are possible within the scope of the present invention. Accordingly, the disclosed preferred best mode embodiments are to be construed as illustrative only. Those who are skilled in the art can make various variations and/or modifications without departing from the scope and spirit of this invention. It should be apparent that features of one embodiment can be combined with one or more features of another embodiment to form a plurality of embodiments. The inventor(s) of the present invention is not required to describe each and every conceivable and possible future embodiment in the preferred best mode embodiments of the present invention. See SRI Int'l v. Matsushita Elec. Corp. of America, 775F.2d 1107, 1121, 227 U.S.P.Q. (BNA) 577, 585 (Fed. Cir. 1985) (enbanc).

The scope and spirit of this invention shall be defined by the claims and the equivalents of the claims only. The exclusive use of all variations and/or modifications within the scope of the claims is reserved. The general presumption is that claim terms should be interpreted using their plain and ordinary meaning without improperly importing a limitation from the specification into the claims. See Continental Circuits LLC v. Intel Corp. (Appeal Number 2018-1076, Fed. Cir. Feb. 8, 2019) and Oxford Immunotec Ltd. v. Qiagen, Inc. et al., Action No. 15-cv-13124-NMG. Unless a claim term is specifically defined in the preferred best mode embodiments, then a claim term has an ordinary meaning, as understood by a person with an ordinary skill in the art, at the time of the present invention. Plain claim language will not be narrowed, unless the inventor(s) of the present invention clearly and explicitly disclaims broader claim scope. See Sumitomo Dainippon Pharma Co. v. Emcure Pharm. Ltd., Case Nos. 17-1798; -1799; -1800 (Fed. Cir. Apr. 16, 2018) (Stoll, J). As noted long ago: “Specifications teach. Claims claim”. See Rexnord Corp. v. Laitram Corp., 274 F.3d 1336, 1344 (Fed. Cir. 2001). The rights of claims (and rights of the equivalents of the claims) under the Doctrine of Equivalents-meeting the “Triple Identity Test” (a) performing substantially the same function, (b) in substantially the same way and (c) yielding substantially the same result. See Crown Packaging Tech., Inc. v. Rexam Beverage Can Co., 559 F.3d 1308, 1312 (Fed. Cir. 2009)) of the present invention are not narrowed or limited by the selective imports of the specifications (of the preferred embodiments of the present invention) into the claims.

While “absolute precision is unattainable” in patented claims, the definiteness requirement “mandates clarity.” See Nautilus, Inc. v. Biosig Instruments, Inc., 527 U.S.______, 134 S. Ct. 2120, 2129, 110 USPQ2d 1688, 1693 (2014). Definiteness of claim language must be analyzed NOT in a vacuum, but in light of:
(a) The content of the particular application disclosure,
(b) The teachings of any prior art and
(c) The claim interpretation that would be given by one possessing the ordinary level of skill in the pertinent art at the time the invention was made. (Id.).
   See Orthokinetics, Inc. v. Safety Travel Chairs, Inc., 806 F.2d 1565, 1 USPQ2d 1081 (Fed. Cir. 1986)

There are number of ways the written description requirement is satisfied. Applicant(s) does not need to describe every claim element exactly, because there is no such requirement (MPEP § 2163). Rather to satisfy the written description requirement, all that is required is “reasonable clarity” (MPEP § 2163.02). An adequate description may be made in any way through express, implicit or even inherent disclosures in the application, including word, structures, figures, diagrams and/or equations (MPEP §§ 2163(1), 2163.02). The set of claims in this invention generally covers a set of sufficient number of embodiments to conform to written description and enablement doctrine. See Ariad Pharm., Inc. v. Eli Lilly & Co., 598 F.3d 1336, 1355 (Fed. Cir. 2010), Regents of the University of California v. Eli Lilly & Co., 119 F.3d 1559 (Fed. Cir. 1997) & Amgen Inc. v. Chugai Pharmaceutical Co. 927 F.2d 1200 (Fed. Cir. 1991).

Furthermore, Amgen Inc. v. Chugai Pharmaceutical Co. exemplifies Federal Circuit's strict enablement requirements. Additionally, the set of claims in this invention is intended to inform the scope of this invention with “reasonable certainty”. See Interval Licensing, LLC v. AOL Inc. (Fed. Cir. Sep. 10, 2014). A key aspect of the enablement requirement is that it only requires that others will not have to perform “undue experimentation” to reproduce it. Enablement is not precluded by the necessity of some experimentation, “[t]he key word is ‘undue’, not experimentation.” Enablement is generally considered to be the most important factor for determining the scope of claim protection allowed. The scope of enablement must be commensurate with the scope of the claims. However, enablement does not require that an inventor disclose every possible embodiment of his invention. The scope of enablement must be commensurate with the scope of the claims. The scope of the claims must be less than or equal to the scope of enablement. See Promega v. Life Technologies Fed. Cir., Dec. 2014, Magsil v. Hitachi Global Storage Fed. Cir. Aug. 2012.

The term “means” was not used nor intended nor implied in the disclosed preferred best mode embodiments of the present invention. Thus, the inventor(s) has not limited the scope of the claims as mean plus function.

An apparatus claim with functional language is not an impermissible “hybrid” claim; instead, it is simply an apparatus claim including functional limitations. Additionally, “apparatus claims are not necessarily indefinite for using functional language . . . [f]unctional language may also be employed to limit the claims without using the means-plus-function format.” See National Presto Industries, Inc. v. The West Bend Co., 76 F. 3d 1185 (Fed. Cir. 1996), R.A.C.C. Indus. v. Stun-Tech, Inc., 178 F.3d 1309 (Fed. Cir. 1998) (unpublished), Microprocessor Enhancement Corp. v. Texas Instruments Inc. & Williamson v. Citrix Online, LLC, 792 F.3d 1339 (2015). The present invention is set forth in the accompanying claims.

I claim:

1. An engineered dendritic cell comprises:
(a) a first bioactive molecule;
wherein the first bioactive molecule is selected from the group consisting of: a co-stimulating molecule, a mobility enhancing molecule and a programming molecule,
(b) an identifying protein on the surface of a cancer cell, or a sensing protein to detect a cancer cell; and
(c) a nanostructure,
wherein the nanostructure comprises a deoxyribonucleic acid (DNA) origami based nanostructure, or a ribonucleic acid (RNA) origami based nanostructure, or a XNA origami based nanostructure for interaction with a T-cell, and/or a natural killer cell against a cancer cell,
wherein XNA comprises genetic bases adenine (A), thymine (T), guanine (G), cytosine (C) and uracil (U), and one or more synthetic or artificial genetic bases,
wherein the nanostructure is encapsulated in a lipid bilayer, or the nanostructure is attached with a red blood cell, or a white blood cell.

2. The engineered dendritic cell in claim 1, further comprises a second bioactive molecule to initiate response of the T-cell, and/or the natural killer cell.

3. The engineered dendritic cell in claim 1, further comprises an activating protein to detect a molecular event within a cancer cell.

4. The engineered dendritic cell in claim 1, further exchanges or transfers the identifying protein, or the sensing protein to the T-cell, and/or the natural killer cell.

5. The engineered dendritic cell in claim 1, further comprises the Ly49 protein, and/or a patient-specific Major Histocompatibility Complex (MHC) protein.

6. The engineered dendritic cell in claim 1, further comprises the Ly49 protein, and/or a synthetic Major Histocompatibility Complex (MHC) molecule, wherein the synthetic Major Histocompatibility Complex (MHC) molecule is further chemically coupled with a peptide of a particular type of cancer cells.

7. The engineered dendritic cell in claim 1, further comprises a first nanoshell, or a second nanoshell, or a third nanoshell, wherein the first nanoshell encapsulates deoxyribonucleic acid (DNA) sequence to encode a therapeutic protein against a cancer cell, wherein the second nanoshell encapsulates XNA sequence to encode a therapeutic protein against a cancer cell, wherein XNA comprises genetic bases adenine (A), thymine (T), guanine (G), cytosine (C) and uracil (U), and one or more synthetic or artificial genetic bases, wherein the third nanoshell comprises a molecule, or a messenger RNA (mRNA) for coding Cas9 and a single-guide RNA (sgRNA).

8. The engineered dendritic cell in claim 7, wherein the first nanoshell, or the second nanoshell, or the third nanoshell comprises disulfide bonds.

9. The engineered dendritic cell in claim 7, wherein the first nanoshell, or the second nanoshell, or the third nanoshell is coupled or chemically coupled with a molecular complex, wherein the molecular complex comprises a long-chain fatty acid with a binding site for a molecule.

10. The engineered dendritic cell in claim 9, wherein the molecular complex is encapsulated within human serum albumin (HSA).

11. The engineered dendritic cell in claim 7, wherein the first nanoshell, or the second nanoshell, or the third nanoshell is a carbon nanodot.

12. The engineered dendritic cell in claim 7, further exchanges or transfers the first nanoshell, or the second nanoshell, or the third nanoshell to the T-cell, and/or the natural killer cell.

13. The engineered dendritic cell in claim 7, further comprises a liposome, wherein the liposome includes a molecule, or a drug and a magnetic nanoparticle, wherein the liposome is heated by a magnetic field.

14. The engineered dendritic cell in claim 13, further exchanges or transfers the first nanoshell, or the second nanoshell, or the third nanoshell to the T-cell, and/or the natural killer cell.

15. The engineered dendritic cell in claim 1, wherein the nanostructure further comprises RNAi based logic circuit.

16. The engineered dendritic cell in claim 1, wherein the nanostructure is functionalized by a pattern of ligands to induce, or activate dendritic cell to initiate response of the T-cell, and/or the natural killer cell.

17. The engineered dendritic cell in claim 1, further comprises a CRISPR-Cas9 system, or a light activated CRISPR-Cas9 system, wherein the CRISPR-Cas9 system, or the light activated CRISPR-Cas9 system removes a first defective gene for inactivation of a dendritic cell, and adds/inserts/replaces a second gene, wherein the second gene expresses, or releases a third bioactive molecule to increase interaction with the T-cell, and/or the natural killer cell.

18. The engineered dendritic cell in claim 17, wherein the second gene comprises one or more synthetic or artificial genetic bases.

19. The engineered/edited dendritic cell in claim 17, wherein the Cas9 is replaced by Cas9-HFI, or Cas12a.

20. The engineered/edited dendritic cell in claim 17, wherein the CRISPR-Cas9 system is replaced by a transposon-encoded CRISPR-Cas system.

21. The engineered dendritic cell in claim 1, further couples with or comprises a modified exosome.

22. The engineered dendritic cell in claim 1, is an engineered and modulated dendritic cell, wherein the engineered and modulated dendritic cell further comprises a modulating CRISPR-Cas9 system, wherein the modulating CRISPR-Cas9 system comprises Cas9, a short single guide RNA, a transcriptional activator and an adeno-associated virus (AAV), wherein the modulating CRISPR-Cas9 system activates an expression of one or more genes beneficial gene against a cancer cell.

23. An engineered dendritic cell comprises:
(a) a first bioactive molecule;
wherein the first bioactive molecule is selected from the group consisting of the following: a co-stimulating molecule, a mobility enhancing molecule and a programming molecule,
(b) an identifying protein on the surface of a cancer cell, or a sensing protein to detect a cancer cell; and
(c) a nanostructure,
wherein the nanostructure comprises a deoxyribonucleic acid (DNA) origami based nanostructure, or a ribonucleic acid (RNA) origami based nanostructure, or a XNA origami based nanostructure for interaction with a T-cell, and/or a natural killer cell against a cancer cell,
wherein XNA comprises genetic bases adenine (A), thymine (T), guanine (G), cytosine (C) and uracil (U), and one or more synthetic or artificial genetic bases,
wherein the nanostructure further comprises RNAi based logic circuit.

24. An engineered dendritic cell, wherein the engineered dendritic cell is residing in a biocompatible, or a biodegradable lymph node,
wherein the engineered dendritic cell comprises:
(a) a first bioactive molecule;
wherein the first bioactive molecule is selected from the group consisting of the following: a co-stimulating molecule, a mobility enhancing molecule and a programming molecule,
(b) an identifying protein on the surface of a cancer cell, or a sensing protein to detect a cancer cell; and
(c) a nanostructure, or a scaffold of biocompatible polymers, or a scaffold of silk-fibroin, wherein the nanostructure comprises a deoxyribonucleic acid (DNA) origami based nanostructure, or a ribonucleic acid (RNA) origami based nanostructure, or a XNA origami based nanostructure for interaction with the T-cell, and/or the natural killer cell against a cancer cell, wherein XNA comprises genetic bases adenine (A), thymine (T), guanine (G), cytosine (C) and uracil (U), and one or more synthetic or artificial genetic bases,
wherein the nanostructure is functionalized by a pattern of ligands to induce, or activate dendritic cell to initiate response of a T-cell, and/or a natural killer cell,
wherein the nanostructure is encapsulated in a lipid bilayer, or the nanostructure is attached with a red blood cell, or a white blood cell.

25. The engineered dendritic cell in claim 24, further comprises the Ly49 protein, and/or a Major Histocompatibility Complex (MHC) protein.

26. The engineered dendritic cell in claim 24, further comprises the Ly49 protein, and/or a synthetic Major Histocompatibility Complex (MHC) molecule, wherein the synthetic Major Histocompatibility Complex molecule is further chemically coupled with a peptide of a particular type of cancer cells.

27. The engineered dendritic cell in claim 24, further exchanges or transfers the identifying protein, or the sensing protein to the T-cell, and/or the natural killer cell.

28. The engineered dendritic cell in claim 24, further comprises a first nanoshell, or a second nanoshell, or a third nanoshell, wherein the first nanoshell encapsulates deoxyribonucleic acid (DNA) sequence to encode a therapeutic protein against a cancer cell, wherein the second nanoshell encapsulates XNA sequence to encode a therapeutic protein against a cancer cell, wherein XNA comprises genetic bases adenine (A), thymine (T), guanine (G), cytosine (C) and uracil (U), and one or more synthetic or artificial genetic bases, wherein the third nanoshell comprises a molecule, or a messenger RNA (mRNA) for coding Cas9 and a single-guide RNA (sgRNA).

29. The engineered dendritic cell in claim 28, wherein the first nanoshell, or the second nanoshell, or the third nanoshell comprises disulfide bonds.

30. The engineered dendritic cell in claim 28, wherein the first nanoshell, or the second nanoshell, or the third nanoshell is coupled or chemically coupled with a molecular complex, wherein the molecular complex comprises a long-chain fatty acid with a binding site for a molecule.

31. The engineered dendritic cell in claim 30, wherein the molecular complex is encapsulated within human serum albumin (HSA).

32. The engineered dendritic cell in claim 28, wherein the first nanoshell, or the second nanoshell, or the third nanoshell is a carbon nanodot.

33. The engineered dendritic cell in claim 28, further exchanges or transfers the first nanoshell, or the second nanoshell, or the third nanoshell to the T-cell, and/or the natural killer cell.

34. The engineered dendritic cell in claim 28, further comprises a liposome, wherein the liposome includes a bioactive molecule, or a drug and a magnetic nanoparticle, wherein the liposome is heated by a magnetic field.

35. The engineered dendritic cell in claim 34, further exchanges or transfers the first nanoshell, or the second nanoshell, or the third nanoshell to the T-cell, and/or the natural killer cell.

36. The engineered dendritic cell in claim 24, wherein the nanostructure further comprises RNAi based logic circuit.

37. The engineered dendritic cell in claim 24, further comprises a CRISPR-Cas9 system, or a light activated CRISPR-Cas9 system, wherein the CRISPR-Cas9 system, or the light activated CRISPR-Cas9 system removes a first defective gene for inactivation of a dendritic cell, and adds/inserts/replaces a second gene, wherein the second gene expresses, or releases a third bioactive molecule to increase interaction with the T-cell, and/or the natural killer cell.

38. The engineered and edited dendritic cell in claim 37, wherein the second gene comprises one or more synthetic or artificial genetic bases.

39. The engineered and edited dendritic cell in claim 37, wherein the Cas9 is replaced by Cas9-HFI, or Cas12a.

40. The engineered and edited dendritic cell in claim 37, wherein the CRISPR-Cas9 system is replaced by a transposon-encoded CRISPR-Cas system

41. The engineered dendritic cell in claim 24, is an engineered and modulated dendritic cell, wherein the engineered and modulated dendritic cell further comprises a modulating CRISPR-Cas9 system, wherein the modulating CRISPR-Cas9 system comprises Cas9, a short single guide RNA, a transcriptional activator and an adeno-associated virus (AAV), wherein the modulating CRISPR-Cas9 system activates an expression of one or more genes beneficial gene against a cancer cell.

42. The engineered dendritic cell in claim 24, wherein the biocompatible, or the biodegradable lymph node is coated with a lattice of microgels.

43. The engineered dendritic cell in claim 24, wherein the biocompatible, or the biodegradable lymph node is dipped in an antibiotic solution.

44. The engineered dendritic cell in claim 24, wherein the biocompatible, or the biodegradable lymph node comprises a metallic glass material.

45. The engineered dendritic cell in claim 24, wherein the biocompatible, or the biodegradable lymph node comprises a mechanically actuating membrane.
